(19) United States
(12) Patent Application Publication
(10) Pub. No.: US 2022/0183316 A1
(43) Pub. Date: Jun. 16, 2022

(54) FOOD PRODUCTS COMPRISING CULTIVATED BOVINE CELLS AND METHODS THEREOF

(71) Applicant: GOOD Meat, Inc., San Francisco, CA (US)

(72) Inventors: Vitor Espirito SANTO, San Francisco, CA (US); Chuong Minh NGUYEN, San Jose, CA (US); Md Amranul HAQUE, Albany, CA (US); Paola BIGNONE, Los Angeles, CA (US)

(21) Appl. No.: 17/551,983

(22) Filed: Dec. 15, 2021

Related U.S. Application Data
(60) Provisional application No. 63/126,158, filed on Dec. 16, 2020.

(51) Int. Cl. A23J 3/04; C12N 5/077

(52) U.S. Cl. CPC A23J 3/04; C12N 5/0652; C12N 2500/46; C12N 2500/24; C12N 2500/90

(57) ABSTRACT

Provided herein are bovine cells that are adapted to grow in growth medium that contains low-serum or no serum and methods thereof. Also provided are food products made from bovine cells cultivated in vitro and methods for harvesting the cells.

### CROSS-REFERENCE TO RELATED APPLICATIONS

This application claims priority from U.S. provisional patent application No. 63/126,158 filed on Dec. 16, 2020. The entire contents of this earlier filed application are hereby incorporated by reference in their entirety.

### FIELD

The present disclosure relates to food products made from bovine cells produced in vitro, in low serum or serum-free growth media, and methods of cultivating bovine cells. Also disclosed are immortalized bovine cells cultivated in low serum or serum-free growth media.

### BACKGROUND

The consumption of beef has been a part of the human diet for thousands of years. Modern domestic cattle (Bos taurus) is believed to have been domesticated 10,000-5,000 years ago. Currently, there are believed to be about 1 billion domestic cattle in the world.

Today, there is an ever-growing demand for meat with a concurrent rise in the human population, which conventional animal agriculture cannot address efficiently (Specht et al., 2018). Therefore, there is an increased interest in cultured meat, also designated as cell-based meat or cultivated meat, which is produced from animal cells using animal cell culture. Cultured beef is a sustainable alternative to traditional livestock-derived beef if challenges around the diversity in types of meat based on the source, cut and breed can be addressed.

The farming of animals for human consumption has significant environmental impacts. In 2006, the United Nations Food and Agricultural Organization estimated that animal farming produces about 18 percent of the total greenhouse gases produced by human activity. The UN estimated that greenhouse gases produced by animal farming exceeded greenhouse gases produced by the entire transportation industry, including greenhouse gases produced by automobiles, trucks, trains, ships, and airplanes combined. Additionally, there are health risks in consuming farmed animals. The slaughter and processing of animals exposes the animal carcasses to microbial contamination and exposes people to potentially deadly microbes that remain on the meat.

In conventional tissue culture, serum from the blood of an animal, typically calf serum or fetal bovine serum is required for the cell to grow. The regulatory and economic rationale for elimination of serum from cell growth medium has been well established (Versteegen R, Bioprocessing J, 2016). The use of animal sera in cell-culture processes brings along with it the potential for introduction of adventitious agents such as viruses and other transmissible agents (e.g., bovine spongiform encephalopathy). Additionally, the use of animal sera as a raw material introduces batch-to-batch variation and impacts negatively on the economics of large-scale cell-culture processes. Several cell culture media formulations are commercially available, including hundreds of commercial products free of animal-derived constituents (Kolkmann et al., 2020). Thus far, cell culture media production for animal cells has been developed towards applications in the biopharmaceutical industry, which does not operate under the same constraints as a food production process. Notably, the production requirements for food applications are likely to be less stringent as for therapeutic or research operations, potentially enabling cost savings resulting from the grade of raw materials and final products. The majority of the commercially available serum-free media are expensive and are limited by proprietary media formulations, but there is a need to develop an in-house defined culture media tailored to promote the growth of specific cell types and cultivation processes for cultured meat manufacturing. For instance, few recent studies have already shown the successful growth of mammalian muscle cells in custom made serum-free media albeit smaller research scale (Sinacore et al., 2000). Recently, Arye et. al., has shown the use of low serum condition for developing cultured meat using plant-based scaffolds (Ben-Arye and Levenberg, 2019).

Cultured meat products have the potential to: (1) substantially reduce reliance on slaughtered animals for food use, (2) lessen the environmental burden of raising animals for food supply, and (3) provide a reliable source of protein that is both safe and has consistent quality.

### SUMMARY

The present disclosure provides cells of the genus Bos, wherein the cells are adapted to grow in a growth medium that comprises low-serum or no serum. In one embodiment, the cells are immortalized cells. In some embodiments, the immortalized cells are non-tumorigenic.

In one embodiment, the cells are adapted to grow in a growth medium comprising serum derived from an animal. The serum in one embodiment is calf serum or fetal bovine serum.

Disclosed herein are muscle cells, myosatellite cells, myoblasts, fat cells, pre-adipocytes or adipocytes of the genus Bos that can be cultivated in a growth medium that comprises low-serum or no-serum.

In an embodiment, disclosed herein are muscle cells or myoblasts wherein endogenous expression of surface receptors is upregulated or downregulated. In one embodiment, the endogenously expressed cell surface receptor is selected from the group consisting of CD29, CD56, and CD82.

In an embodiment, disclosed herein are muscle cells or myoblast cells wherein the endogenous expression of cell transcription factors is upregulated. In one embodiment, the upregulated endogenously expressed transcription factor is selected from the group consisting of PAX3, PAX7, Myf5, Mrf4, MyoD, and MyoG.

Another embodiment disclosed herein are muscle cells or myoblast cells wherein the endogenous expression of desmin or myosin heavy chain 2 (MyHC2) is upregulated.

In an embodiment, disclosed herein are fat cells, pre-adipocytes or adipocyte cells wherein the endogenous expression of cell surface receptors, transcription factors, or other gene products is upregulated or downregulated.

In an embodiment, disclosed herein are preadipocytes cells wherein the endogenous expression of Pref-1, C/EBP beta, C/EBP gamma, PPAR, or C/EBP alpha is upregulated.

In an embodiment, in the fat cells or adipocytes disclosed herein, the endogenous expression of PPAR gamma, C/EBP alpha, adiponectin, lipoprotein lipase, or FABP4 is upregulated.

In one embodiment, the cells provided herein are cells of the genus Bos that are engineered to express a telomerase reverse transcriptase (TERT). In an embodiment, the cells are transduced to express a bovine telomerase reverse transcriptase (bTERT).

The present disclosure provides methods of cultivating cells of the genus Bos, wherein the cells are adapted to grow in a growth medium that comprises low-serum or no serum. In one embodiment, the cells are immortalized cells. In some embodiments, the immortalized cells are non-tumorigenic.

In one embodiment, the method cultivates cells that are adapted to grow in a growth medium comprising serum derived from an animal. The serum in one embodiment is calf serum or fetal bovine serum.

Disclosed herein are methods of cultivating muscle cells, myosatellite cells, myoblasts, fat cells, pre-adipocytes, or adipocytes of the genus Bos in a growth medium that comprises low-serum or no-serum.

In an embodiment, the methods disclosed herein cultivate muscle cells or myoblasts, wherein the endogenous expression of cell surface receptors is upregulated or downregulated. In one embodiment, the endogenously expressed cell surface receptor is selected from the group consisting of CD29, CD56, and CD82.

In an embodiment, in the methods of cultivating muscle cells or myoblast cells the cells endogenously express cell transcription factors. In one embodiment, the muscle cells or myoblasts cells manufactured by the methods described herein, are cells wherein the endogenous expression of transcription factor selected from the group consisting of Pax3, Pax7, Myf5, Mrf4, MyoD, and MyoG is upregulated.

Another embodiment disclosed herein are methods of cultivating muscle cells or myoblasts, wherein the endogenous expression of desmin or myosin heavy chain 2 (MyHC2) is upregulated.

In an embodiment, the methods provided herein are used to cultivate fat cells, pre-adipocytes or adipocyte cells wherein the endogenous expression of cell surface receptors, transcription factors, or other gene products is upregulated or downregulated.

In an embodiment, the methods provided herein produce preadipocytes cells wherein the endogenous expression of Pref-1, C/EBP beta, C/EBP gamma, PPAR, or C/EBP alpha is upregulated.

In an embodiment, the methods provided herein are used to cultivate fat cells, or adipocytes wherein the expression of PPARgamma, C/EBPalpha, adiponectin, lipoprotein lipase, or FABP4 is upregulated.

In one embodiment the methods provided herein cultivates cells of the genus Bos that are engineered to express a telomerase reverse transcriptase (TERT). In an embodiment, the cells are transduced to express a bovine telomerase reverse transcriptase (bTERT)

In some embodiments, the growth medium provided herein can comprise one or more of growth factors, fatty acids, proteins, elements, and small molecules.

In some embodiments, the growth factor is selected from the group consisting of insulin growth factor, fibroblast growth factor, and epidermal growth factor.

In some embodiments, the growth media comprises transferrin.

In some embodiments, the growth media comprises selenium.

In some embodiments, the growth media comprises ethanolamine.

In some embodiments, the cells disclosed herein are cultivated in adherent cultures or in suspension cultures.

The present disclosure also provides compositions for food products that comprise cultivated Bos cells. This disclosure also sets forth processes for making and using products.

In some embodiments, there are provided methods of producing a food product comprising cells of the genus Bos cultured in vitro, the methods comprising culturing a population of cells in vitro in a growth medium capable of maintaining the cells, recovering cells, and formulating the recovered cells into an edible food product. In some embodiments, the cells comprise muscle cells, myosatellite cells, myoblasts, fat cells, pre-adipocytes, or adipocytes.

In some embodiments, there are provided methods of preparing a food product made from cells of the genus Bos grown in vitro, the method comprising the steps of: conditioning water with a phosphate to prepare conditioned water, hydrating a plant protein isolate or plant protein concentrate with the conditioned water to produce hydrated plant protein, contacting the cell paste with the hydrated plant protein to produce a cell and pulse protein mixture, heating the cell and plant protein mixture in steps, wherein the steps comprise at least one of:

ramping up the temperature of the cell and protein mixture to a temperature between 40-65° C., maintaining the temperature of the cell and protein mixture at a temperature between 40-65° C. for 1 to 30 minutes, ramping up the temperature of the cell and protein mixture to a temperature between 60-85° C., cooling the cell and protein mixture to a temperature between −1-25° C., and admixing the cell and protein mixture with a fat to create a pre-cooking product. The pre-cooking product can be consumed without further cooking. Alternatively, the pre-cooking product is cooked to produce the edible food product. Optionally, the pre-cooking product may be stored at room temperature, refrigeration temperatures or frozen.

In some embodiments, there are provided food products produced from cells of the genus Bos, comprising a cell paste, the cell paste content of at least 5% by weight, and wherein the cell paste is made from cells grown in vitro; a plant protein isolate or plant protein concentrate, the plant protein content at least 5% by weight; a fat, the fat content at least 5% by weight; and water, the water content at least 5% by weight.

In some embodiments, the food composition or food product comprises about 1%-100% by weight wet cell paste.

In some embodiments, plant protein isolates or plant protein concentrates are obtained from pulses selected from the group consisting of dry beans, lentils, mung beans, fava beans, dry peas, chickpeas, cowpeas, bambara beans, pigeon peas, lupins, vetches, adzuki, common beans, fenugreek, long beans, lima beans, runner beans, or tepary beans, soybeans, or mucuna beans. In various embodiments, the pulse protein isolates or plant protein concentrates provided herein are derived from Vigna angularis, Vicia faba, Cicer arietinum, Lens culinaris, Phaseolus vulgaris, Vigna unguiculata, Vigna subterranea, Cajanus cajan, Lupinus sp., Vetch sp., Trigonella foenum-graecum, Phaseolus lunatus, Phaseolus coccineus, or Phaseolus acutifolius. In some embodiments, the pulse protein isolates are derived from mung beans. In some embodiments, the mung bean is Vigna radiata.

In some embodiments, animal protein isolate and animal protein concentrate are obtained from animals or animal products. Examples of animal protein isolate or animal protein concentrate include whey, casein, and egg protein.

In some embodiments, plant protein isolates are obtained from wheat, rice, teff, oat, corn, barley, sorghum, rye, millet, triticale, amaranth, buckwheat, quinoa, almond, cashew, pecan, peanut, walnut, macadamia, hazelnut, pistachio, brazil, chestnut, kola nut, sunflower seeds, pumpkin seeds, flax seeds, cacao, pine nut, ginkgo, and other nuts.

### BRIEF DESCRIPTIONS OF THE DRAWINGS

FIG. 1 shows the proliferation of B4M myoblasts.

FIG. 2 provides a phase contrast microscopic image showing the differentiation of myoblasts into myotubes/myofibers.

FIG. 3 shows a schematic representation of expression lentivirus construct for expression of bTERT.

FIG. 4 shows the proliferation of immortalized B4M cells (B4M-t6). B4M-t1 are cells that are not transduced to express bTERT and used as controls during transduction.

FIG. 5 shows the viable cell density of B4M-t6 cells during adaptation to suspension cultures.

FIG. 6 shows population doubling times of B4M-t6 cells adapted to grow in media without supplementation with fetuin or fetal bovine serum.

FIG. 7 shows the viable cell density of B10M-t3 cells during adaptation to suspension culture.

FIG. 8a shows a schematic representation of a sleeping beauty vector construct for expression of bTERT with antibiotic selection. FIG. 8b shows a schematic representation of a sleeping beauty vector construct for expression of bTERT without antibiotic selection.

FIG. 9 shows the proliferation of immortalized B9M cells (B9M-SB3 and B9M-SB10). B9M 1 and B9M 2 are independent populations of cells that are not transfected with Sleeping Beauty vectors to express bTERT and used as controls during transfection.

FIG. 10a shows a microscopic image (10× magnification) of the B9M cells that are not transfected with Sleeping Beauty vectors to express bTERT. FIG. 10b shows B9M-SB3 cells (B9M-tert) that are immortalized with Sleeping Beauty vectors (B) to express bTERT at the 170th day of culture.

### DETAILED DESCRIPTION

The following description is presented to enable one of ordinary skill in the art to make and use the disclosed subject matter and to incorporate it in the context of applications. Various modifications, as well as a variety of uses in different applications, will be readily apparent to those skilled in the art, and the general principles defined herein may be applied to a wide range of embodiments. Thus, the present disclosure is not intended to be limited to the embodiments presented but is to be accorded the widest scope consistent with the principles and novel features disclosed herein.

### Definitions

As used herein, the term “batch culture” refers to a closed culture system with nutrient, temperature, pressure, aeration, and other environmental conditions to optimize growth. Because nutrients are not added, nor waste products removed during incubation, batch cultures can complete a finite number of life cycles before nutrients are depleted and growth stops.

As used herein, the term “edible food product” refers to a food product safe for human consumption. For example, this includes, but is not limited to a food product that is generally recognized as safe per a government or regulatory body (such as the United States Food and Drug Administration). In certain embodiments, the food product is considered safe to consume by a person of skill. Any edible food product suitable for a human consumption should also be suitable for consumption by another animal and such an embodiment is intended to be within the scope herein.

As used herein, the term “enzyme” or “enzymatically” refers to biological catalysts. Enzymes accelerate, or catalyze, chemical reactions. Enzymes increase the rate of reaction by lowering the activation energy.

As used herein, the term “expression” is the process by which information from a gene is used in the synthesis of a functional gene product. As used herein, the term “endogenously expressed” means that the cell expresses a gene that is naturally present in the cell without genetic manipulation.

As used herein, the term “downregulated” means that the expression of a gene in a cell is decreased. For example, when myosatellite cells are differentiated or activated into muscle cells or myoblasts, the expression of certain genes is downregulated in the muscle cells or the myoblasts as compared to myosatellite cells. Similarly, for example, when preadipocytes differentiate into fat cells or adipocytes, the expression of certain genes is downregulated in the fat cells or adipocytes as compared to preadipocyte cells.

As used herein, the term “upregulated” means that the expression of a gene in a cell is increased. For example, when myosatellite cells are differentiated or activated into muscle cells or myoblasts, the expression of certain genes is upregulated in the muscle cells or the myoblasts as compared to myosatellite cells. Similarly, for example, when preadipocytes differentiate into fat cells or adipocytes, the expression of certain genes is upregulated in the fat cells or adipocytes as compared to preadipocyte cells.

As used herein, the term “exogenous expression,” “exogenously expressed,” or the like also means that a gene that is not naturally present in an un-engineered cell (host cell) is expressed in the host cell by introducing one or more copies of a recombinant gene into the host cell. As used herein, the term “exogenous expression,” “exogenously expressed,” or the like also means that a gene that is naturally present in an un-engineered cell (host cell) is expressed in a host cell by introducing one or more copies of a recombinant gene into the host cell.

As used herein, the term “knock-in” refers to an engineered cell, or a method to produce an engineered cell, in which an exogenous gene is introduced into the host cell.

As used herein, the term “knock-out” refers to an engineered cell, or a method to produce an engineered cell, in which a gene that is naturally present in the host cell is (endogenous gene) is deleted, or altered in a manner to prevent or reduce expression of the endogenous gene.

As used herein, the term “myosatellite cell” is a muscle stem cell that is multipotent and can differentiate into mature muscle cells. Myosatellite cells are cells wherein the expression of a gene product selected from the group consisting of CD56, PAX7 and PAX3 is upregulated as compared to the expression the gene product in mature muscle cells or myoblasts. Alternatively, myosatellite cells are cells wherein the expression of a gene product selected from the group consisting of desmin, myosin heavy chain is downregulated as compared to the expression the gene product in mature muscle cells.

As used herein, the term “muscle cell” or “myoblast” is a cell that is not a myosatellite cell. Muscle cells or myoblasts are cells wherein the expression of a gene product selected from the group consisting of MyoD, HGF, and FGF2 is upregulated as compared to the expression the gene product in myosatellite cells. Alternatively, muscle cells are cells wherein the expression of a gene product selected from the group consisting of Notch, Foxo, and miR31 is downregulated as compared to the expression the gene product in myosatellite cells.

As used herein, the term “fat cells” or “adipocytes” are cells that specialize in storing energy as fat. Fat cells or adipocytes are cells wherein the expression of a gene product selected from the group consisting of Adiponectin, lipoprotein lipase, and FABP4 is upregulated as compared to the expression the gene product in preadipocytes or fat stem cells. Alternatively, fat cells or adipocytes cells are cells wherein the expression of a gene product selected from the group consisting of PPARγ, CEBPα, SREBP, Zfp423, GATA3, Wnt10b, Wnt10a, Wnt6, Mmp3, and Twist2 is downregulated as compared to the expression the gene product in preadipocytes of fat stem cells.

As used herein, the term “preadipocytes” or “fat stem cells” are cell capable of differentiating into fat cells or adipocytes. Preadipocytes or fat stem cells cells wherein the expression of a gene product selected from the group consisting of PPAR gamma (PPARγ), CEBP alpha (CEBPα), SREBP, Zfp423, GATA3, Wnt10b, Wnt10a, Wnt6, Mmp3, and Twist2 is upregulated as compared to the expression the gene product in fat cells of adipocytes. Alternatively, preadipocytes or fat stem cells are cells wherein the expression of a gene product selected from the group consisting of Adiponectin, lipoprotein lipase, and FABP4 is downregulated as compared to the expression the gene product in fat cells or adipocytes.

As used herein, the term the term “non-tumorigenic” means a cell that does not express a family of genes that belong to pathways described to trigger formation or growth of tumors, including but not limited to pathways implicated in cancer (KEGG_05200), transcriptional misregulation in cancer (KEGG_05202), microRNAs in cancer (KEGG_05206), proteoglycans in cancer (KEGG_05205), chemical carcinogenesis (KEGG_05204), viral carcinogenesis (KEGG_05203), central carbon metabolism in cancer (KEGG_05230), choline metabolism in cancer (KEGG_05231) and PD-L1 expression and PD-1 checkpoint pathway in cancer (KEGG_05235).

As used herein, the term “immortalized cell” is a cell that can be propagated in vitro for more than 60 population doublings and in the case of some cell lines, they can be propagated indefinitely.

As used herein a cell surface receptor is a protein that is expressed on the surface of cells. Cell types of different lineages express different cell surface receptors.

As used herein a transcription factor is a protein expressed by a cell that regulates the expression of genes. Cell types of different lineages express different transcription factors.

As used herein, “desmin” and “myosin” are proteins expressed by committed and/or differentiated muscle cell. “Myosin heavy chain 2” (MyHC2) is a fibrous protein that is expressed by a differentiated muscle cell.

As used herein, the term “telomerase reverse transcriptase,” or “TERT” is the catalytic subunit of the enzyme telomerase. Telomerase lengthens the telomeres of chromosomes strains leading to inhibition of apoptosis.

As used herein, a bovine of the Bos genus is an animal that is farmed for human consumption. Species of Bos include B. buiaensis, B. frontails, B. grunniens, B. javanicus, B. savueli, and B. taurus.

As used herein, the term “fed-batch culture” refers to an operational technique where one or more nutrients, such as substrates, are fed to a bioreactor in continuous or periodic mode during cultivation and in which product(s) remain in the bioreactor until the end of a run. An alternative description is that of a culture in which a base medium supports initial cell culture and a feed medium is added to prevent nutrient depletion. In a fed-batch culture one can control concentration of fed-substrate in the culture liquid at desired levels to support continuous growth.

As used herein the term “small molecule” is a molecule that has a molecular weight of less than 5,000 Dalton.

As used herein, a “gene product” is the biochemical material, either RNA or protein, resulting from expression of a gene.

As used herein, “growth medium” refers to a medium or culture medium that supports the growth of microorganisms or cells or small plants. A growth medium may be, without limitation, solid or liquid or semi-solid. Growth medium shall also be synonymous with “growth media.”

As used herein, “basal medium” refers to a non-supplemented medium which promotes the growth of many types of microorganisms and/or cells which do not require any special nutrient supplements.

As used herein, “in vitro” refers to a process performed or taking place in a test tube, culture dish, bioreactor, or elsewhere outside a living organism. In the body of this disclosure, a product may also be referred to as an in vitro product, in which case in vitro shall be an adjective and the meaning shall be that the product has been produced with a method or process that is outside a living organism.

As used herein, “suspension culture” refers to a type of culture in which single cells or small aggregates of cells multiply (grow) while suspended in agitated liquid medium. It also refers to a cell culture or a cell suspension culture.

As used herein, “adherent culture” refers to a type of culture in which cells can propagate or multiply (grow) while adhered to the surface of a flask or other scaffold. The scaffold is any object that provides a surface on to which the cells adhere. The scaffold can be an edible object, for example but not limited to an extruded protein or an extruded cell.

As used herein, “cell paste” refers to a paste of cells harvested from a cell culture that contains water. The dry cell weight of cell paste can be 1%-5%, 5%-10%, 10%-15%, 15%-20%, 20%-25%, 25%-30%, 30%-35%, 35%-40%, 40%-45%, 45%-50%, or higher. A skilled worker can prepare cell paste with a desired water content. Typically, cell paste comprises about 5%-15% cells by dry cell weight. It is within the ambit of skilled practitioners to prepare cell paste that comprises a desired dry cell weight of cultivated cells, including cell paste that comprises any other desired percentage by dry cell weight. The skilled worker can remove moisture by centrifugation, lyophilization, heating or any other well-known drying techniques. According to the United States Department of Agriculture, the naturally occurring moisture content of animal meats including beef, is about 75% water. In some embodiments, the cell paste provided herein comprises a significant amount of water. “Wet cell paste” as used herein comprises about 25%-90% water 25%-85% water, 25%-80% water, 25%-75% water, 25%-70% water, 25%-65% water, 25%-60% water, 25%-55% water, 25%-50% water, 30%-90% water, 30%-85% water, 30%-80% water, 30%-75% water, 30%-70% water, 30%-65% water, 30%-60% water, 30%-55% water, 30%-50% water, 35%-90% water, 35%-85% water, 35%-80% water, 35%-75% water, 35%-70% water, 35%-65% water, 35%-60% water, 35%-55% water, 35%-50% water, 40%-90% water, 40%-85% water, 40%-80% water, 40%-75% water, 40%-70% water, 40%-65% water, 40%-60% water, 40%-60% water, 40%-55% water, 40%-50% water, 45%-90% water, 45%-85% water, 45%-80% water, 45%-75% water, 45%-70% water, 45%-75% water, 45%-70% water, 45%-65% water, 45%-60% water, 45%-55% water, 45%-50% water, 50%-90% water, 50%-85% water, 50%-80% water, 50%-75% water, 50%-70% water, 50%-65% water, 50%-60% water, 50%-55% water. Cell paste is another term for cultured cell meat.

As used herein, “substantially pure” refers to cells that are at least 80% cells by dry weight. Substantially pure cells are between 80%-85% cells by dry weight, between 85%-90% cells by dry weight, between 90%-92% cells by dry weight, between 92%-94% cells by dry weight, between 94%-96% cells by dry weight, between 96%-98% cells by dry weight, between 98%-99% cells by dry weight.

As used herein, “seasoning” refers to one or more herbs and spices in both solid and liquid form.

As used herein, “primary cells” refer to cells from a parental animal that maintain growth in a suitable growth medium, for instance under controlled environmental conditions. Cells in primary culture have the same karyotype (number and appearance of chromosomes in the nucleus of a eukaryotic cell) as those cells in the original tissue.

As used herein, “secondary cells” refers to primary cells that have undergone a genetic transformation and become immortalized allowing for indefinite proliferation.

As used herein, “proliferation” refers to a process that results in an increase in the number of cells. It is characterized by a balance between cell division and cell loss through cell death or differentiation.

As used herein, “adventitious” refers to one or more contaminants such as, but not limited to: viruses, bacteria, mycoplasma, and fungi.

As used herein “peptide cross-linking enzyme” or “cross-linking enzyme is an enzyme that catalyzes the formation of covalent bonds between one or more polypeptides.

As used herein, “transglutaminase” or “TG” refers to an enzyme (R-glutamyl-peptide amine glutamyl transferase) that catalyzes the formation of a peptide (amide) bond between γ-carboxyamide groups and various primary amines, classified as EC 2,3.2,13. Transglutaminases catalyze the formation of covalent bonds between polypeptides, thereby cross-linked polypeptides. Cross-linking enzymes such as transglutaminase are used in the food industry to improve texture of some food products such as dairy, meat and cereal products. It can be isolated from a bacterial source, a fungus, a mold, a fish, a mammal, or a plant.

As used herein “protein concentrate” is a collection of one or more different polypeptides obtained from a plant source or animal source. The percent protein by dry weight of a protein concentrate is greater than 25% protein by dry weight.

As used herein “protein isolate” is a collection of one or more different polypeptides obtained from a plant source or an animal source. The percent protein by dry weight of a protein concentrate is greater than 50% protein by dry weight.

As used herein, and unless otherwise indicated, percentage (%) refers to total % by weight typically on a dry weight basis unless otherwise indicated.

The term “about” indicates and encompasses an indicated value and a range above and below that value. In certain embodiments, the term “about” indicates the designated value ±10%, ±5%, or ±1%. In certain embodiments, the term “about” indicates the designated value±one standard deviation of that value.

In this disclosure, methods are presented for culturing Bos taurus cells in vitro. The methods herein provide methods to proliferate, recover, and monitor the purity of cell cultures. The cells can be used, for example, in one or more food products.

The disclosure herein sets forth embodiments for food products compositions comprising Bos taurus cells grown in vitro. In some embodiments, the compositions comprise plant protein, cell paste, fat, water, and a peptide cross-linking enzyme.

The disclosure herein sets forth embodiments for methods to prepare a food product made from Bos taurus cells grown in vitro. The food product is an edible food product.

### Cells

Provided herein are food products or processes comprising cells of the genus Bos. In some embodiments, the cells are Bos taurus cells. In some embodiments, the cells are selected from, but not limited to Bos taurus breeds: Angus, Charolais, Hereford, Simmental, Longhorn, Gelbvieh, Holstein, Limousin, Highlands, and Wagyu. In some embodiments, the cells comprise primary Bos taurus cells. In some embodiments, the cells comprise secondary Bos taurus cells.

In some embodiments, the cell lines are immortalized. In some embodiments, the cell lines have high proliferation rates.

In some embodiments, the cells are not recombinant or engineered in any way (i.e., non-GMO). In some embodiments, the cells have not been exposed to any viruses and/or viral DNA. In certain embodiments, the cells are both not recombinant or have not been exposed to any viruses and/or viral DNA and/or RNA.

### Culture Media And Growth

In some embodiments, proliferation occurs in suspension or adherent conditions, with or without feeder-cells and/or in serum-containing or serum-free media conditions. In some embodiments, media for proliferation contains one or more of amino acids, peptides, proteins, carbohydrates, essential metals, minerals, vitamins, buffering agents, anti-microbial agents, growth factors, and/or additional components.

In some embodiments, proliferation is measured by any method known to one skilled in the art. In some embodiments, proliferation is measured through direct cell counts. In certain embodiments, proliferation is measured by a haemocytometer. In some embodiments, proliferation is measured by automated cell imaging. In certain embodiments, proliferation is measured by a Coulter counter.

In some embodiments, proliferation is measured by using viability stains. In certain embodiments, the stains used comprise trypan blue.

In some embodiments, proliferation is measured by the total DNA. In some embodiments, proliferation is measured by BrdU labelling. In some embodiments, proliferation is measured by metabolic measurements. In certain embodiments, proliferation is measured by using tetrazolium salts. In certain embodiments, proliferation is measured by ATP-coupled luminescence.

In some embodiments, the culture media is basal media. In some embodiments, the basal media is SKGM, DMEM, DMEM/F12, MEM, HAMS's F10, HAM's F12, IMDM, McCoy's Media and RPMI.

In some embodiments, the basal media comprises amino acids. In some embodiments, the basal media comprises biotin. In some embodiments, the basal media comprises choline chloride. In some embodiments, the basal media comprises D-calcium pantothenate. In some embodiments, the basal media comprises folic acid. In some of embodiments, the basal media comprises niacinamide. In some embodiments, the basal media comprises pyridoxine hydrochloride. In some embodiments, the basal media comprises riboflavin. In some embodiments, thiamine hydrochloride is part of the basal media (DMEM/F12). In some embodiments, the basal media comprises vitamin B12 (also known as cyanocobalamin). In some embodiments, the basal media comprises i-inositol (myo-inositol). In some embodiments, the basal media comprises calcium chloride. In some embodiments, the basal media comprises cupric sulfate. In some embodiments, the basal media comprises ferric nitrate. In some embodiments, the basal media comprises magnesium chloride. In some embodiments, the basal media comprises magnesium sulfate. In some embodiments, the basal media comprises potassium chloride. In some embodiments, the basal media comprises sodium bicarbonate. In some embodiments, the basal media comprises sodium chloride. In some embodiments, the basal media comprises sodium phosphate dibasic. In some embodiments, the basal media comprises sodium phosphate monobasic. In some embodiments, the basal media comprises zinc sulfate. In some embodiments, the growth medium comprises sugars. In some embodiments, the sugars include but are not limited to D-glucose, galactose, fructose, mannose, or any combination thereof. In an embodiment, the sugars include both D-glucose and mannose. In embodiments where glucose and mannose are both used in the growth medium to cultivate cells, the amount of glucose in the growth medium (cultivation media) is between 0.1-10 g/L, 0.1-9 g/L, 0.1-8 g/L, 0.1-7 g/L, 0.1-6 g/L, 0.1-5 g/L, 0.1-4 g/L, 0.1-3 g/L, 0.1-2 g/L, 0.1-1g/L, 0.5-10 g/L, 0.5-9 g/L, 0.5-8 g/L, 0.5-7 g/L, 0.5-6 g/L, 0.5-5 g/L, 0.5-4 g/L, 0.5-3 g/L, 0.5-2 g/L, 0.5-1 g/L, 1-10 g/L, 1-9 g/L, 1-8 g/L, 1-9 g/L, 1-8 g/L, 1-7 g/L, 1-6 g/L, 1-5 g/L, 1-4 g/L, 1-3 g/L, 1-2 g/L, 2-10 g/L, 2-9 g/L, 2-8 g/L, 2-9 g/L, 2-8 g/L, 2-7 g/L, 2-6 g/L, 2-5 g/L, 2-4 g/L, 2-3 g/L, 3-10 g/L, 3-9 g/L, 3-8 g/L, 3-9 g/L, 3-8 g/L, 3-7 g/L, 3-6 g/L, 3-5 g/L, 3-4 g/L, 4-10 g/L, 4-9 g/L, 4-8 g/L, 4-9 g/L, 4-8 g/L, 4-7 g/L, 4-6 g/L, 4-5 g/L, 5-10 g/L, 5-9 g/L, 5-8 g/L, 5-9 g/L, 5-8 g/L, 5-7 g/L, or 5-6 g/L, and the amount of mannose in the growth media is between 0.1-10 g/L, 0.1-9 g/L, 0.1-8 g/L, 0.1-7 g/L, 0.1-6 g/L, 0.1-5 g/L, 0.1-4 g/L, 0.1-3 g/L, 0.1-2 g/L, 0.1-1g/L, 0.5-10 g/L, 0.5-9 g/L, 0.5-8 g/L, 0.5-7 g/L, 0.5-6 g/L, 0.5-5 g/L, 0.5-4 g/L, 0.5-3 g/L, 0.5-2 g/L, 0.5-1 g/L, 1-10 g/L, 1-9 g/L, 1-8 g/L, 1-9 g/L, 1-8 g/L, 1-7 g/L, 1-6 g/L, 1-5 g/L, 1-4 g/L, 1-3 g/L, 1-2 g/L, 2-10 g/L, 2-9 g/L, 2-8 g/L, 2-9 g/L, 2-8 g/L, 2-7 g/L, 2-6 g/L, 2-5 g/L, 2-4 g/L, 2-3 g/L, 3-10 g/L, 3-9 g/L, 3-8 g/L, 3-9 g/L, 3-8 g/L, 3-7 g/L, 3-6 g/L, 3-5 g/L, 3-4 g/L, 4-10 g/L, 4-9 g/L, 4-8 g/L, 4-9 g/L, 4-8 g/L, 4-7 g/L, 4-6 g/L, 4-5 g/L, 5-10 g/L, 5-9 g/L, 5-8 g/L, 5-9 g/L, 5-8 g/L, 5-7 g/L, or 5-6 g/L. The skilled worker will understand that combinations of these amounts of glucose and mannose can be used, for example, between 2-5 grams of glucose and 1-4 grams of mannose.

In some embodiments, the basal media comprises linoleic acid. In some embodiments, the basal media comprises lipoic acid. In some embodiments, the basal media comprises putrescine-2HCl. In some embodiments, the basal media comprises 1,4 butanediamine. In some embodiments, the basal media comprises Pluronic F-68. In some embodiments, the basal media comprises fetal bovine serum. In certain embodiments, the basal media comprises each ingredient in this paragraph. In certain embodiments, the basal media is DMEM/F12.

In some embodiments, the growth medium comprises serum. In some embodiments, the serum is selected from bovine calf serum, and any combination thereof.

In some embodiments, the growth medium comprises at least 10% fetal bovine serum. In certain embodiments, the population of Bos taurus cells are grown in a medium with at least 10% fetal bovine serum, followed by a reduction to less than 2% fetal bovine serum before recovering the cells, or no fetal bovine serum.

In another embodiment, the culture media contains no serum including fetal bovine serum, fetal calf serum, or any animal derived serum.

In another embodiment, the culture media contains low serum including fetal bovine serum, fetal calf serum, or any animal derived serum. In certain embodiments, low serum comprises less than 5% bovine serum, fetal calf serum, or any animal derived serum before recovering the cells. In certain embodiments, low serum comprises less than 3% bovine serum, fetal calf serum, or any animal derived serum before recovering the cells. In certain embodiments, low serum comprises less than 1% bovine serum, fetal calf serum, or any animal derived serum before recovering the cells.

In certain embodiments, the serum (e.g., fetal bovine serum or fetal calf serum) is reduced to less than or equal to 1.9% serum before recovering the cells. In certain embodiments, the serum is reduced to less than or equal to 1.7% serum before recovering the cells. In certain embodiments, the serum is reduced to less than or equal to 1.5% serum before recovering the cells. In certain embodiments, the serum is reduced to less than or equal to 1.3% serum before recovering the cells. In certain embodiments, the serum is reduced to less than or equal to 1.1% serum before recovering the cells. In certain embodiments, the serum is reduced to less than or equal to 0.9% serum before recovering the cells. In certain embodiments, the serum is reduced to less than or equal to 0.7% serum before recovering the cells. In certain embodiments, the serum is reduced to less than or equal to 0.5% serum before recovering the cells. In certain embodiments, the serum is reduced to less than or equal to 0.3% serum before recovering the cells. In certain embodiments, the serum is reduced to less than or equal to 0.1% serum before recovering the cells. In certain embodiments, the serum is reduced to less than or equal to 0.05% serum before recovering the cells. In certain embodiments, the serum is reduced to about 0% serum before recovering the cells.

In some embodiments, the basal media is DMEM/F12 and is in a ratio of 3:1; 2:1; 1:1, 1:2, or 1:3. In certain embodiments, the basal media is DMEM/F12 and in a ratio of about 3:1, 2:1; 1:1, 1:2, or 1:3.

In some embodiments, the basal media is IMDM/F12 and is in a ratio of 3:1; 2:1; or 1:1, 1:2, or 1:3.

In some embodiments, the growth media is modified in order to optimize the expression of at least one gene from a cell signaling pathway selected from the group consisting of proteasome, steroid biosynthesis, amino acid degradation, amino acid biosynthesis, drug metabolism, focal adhesion, cell cycle, MAPK signaling, glutathione metabolism, TGF-beta, phagosome, terpenoid biosynthesis, DNA replication, glycolysis, gluconeogenesis, protein export, butanoate metabolism, and synthesis and degradation of ketone bodies.

In some embodiments, one or more of the maintenance, proliferation, differentiation, lipid accumulation, lipid content, proneness to purification and/or harvest efficiency, growth rates, cell densities, cell weight, resistance to contamination, expression of endogenous genes and/or protein secretion, shear sensitivity, flavor, texture, color, odor, aroma, gustatory quality, nutritional quality, minimized growth-inhibitory byproduct secretion, and/or minimized media requirements, of Bos taurus cells, in any culture conditions, are improved by the use of one or more of growth factors, proteins, peptides, fatty acids, elements, small molecules, plant hydrolysates, directed evolution, genetic engineering, media composition, bioreactor design, and/or scaffold design. In certain embodiments, the fatty acids comprise stearidonic acid (SDA). In certain embodiments, the fatty acids comprise linoleic acid. In certain embodiments, the growth factor comprises insulin or insulin like growth factor. In certain embodiments, the growth factor comprises fibroblast growth factor or the like. In certain embodiments, the growth factor comprises epidermal growth factor or the like. In certain embodiments, the protein comprises transferrin. In certain embodiments, the element comprises selenium. In certain embodiments, a small molecule comprises ethanolamine. In certain embodiments, a small molecule comprises a steroid or a corticosteroid. In certain embodiments, a small molecule comprises dexamethasone. In certain embodiments, a small molecule comprises ethanolamine. In certain embodiments, the growth medium comprises blood proteins or plasma proteins. In certain embodiments blood protein is fetuin. The amount of ethanolamine used in the cultivations is between 0.05-10 mg/L, 0.05-10 mg/L, 0.1-10 mg/L, 0.1-9.5 mg/L, 0.1-9 mg/L, 0.1-8.5 mg/L, 0.1-8.0 mg/L, 0.1-7.5 mg/L, 0.1-7.0 mg/L, 0.1-6.5 mg/L, 0.1-6.0 mg/L, 0.1-5.5 mg/L, 0.1-5.0 mg/L, 0.1-4.5 mg/L, 0.1-4.0 mg/L, 0.1-3.5 mg/L, 0.1-3.0 mg/L, 0.1-2.5 mg/L, 0.1-2.0 mg/L, 0.1-1.5 mg/L, and 0.1-1.0 mg/L.

In certain embodiments, the media can be supplemented with plant hydrolysates. In certain embodiments, the hydrolysates comprise yeast extract, wheat peptone, rice peptone, phytone peptone, yeastolate, pea peptone, soy peptone, pea peptone, potato peptone, mung bean protein hydrolysate, or sheftone. The amount of hydrolysate used in the cultivations is between 0.1 g/L to 5 g/L, between 0.1 g/L to 4.5 g/L, between 0.1 g/L to 4 g/L, between 0.1 g/L to 3.5 g/L, between 0.1 g/L to 3 g/L, between 0.1 g/L to 2.5 g/L, between 0.1 g/L to 2 g/L, between 0.1 g/L to 1.5 g/L, between 0.1 g/L to 1 g/L, or between 0.1 g/L to 0.5 g/L.

In some embodiments, a small molecule comprises lactate dehydrogenase inhibitors. As described in the Examples below, lactate dehydrogenase inhibitors inhibit the formation of lactate. The production of lactate by the cells inhibits the growth of the cells. Exemplary lactate dehydrogenase inhibitors are selected from the group consisting of oxamate, galloflavin, gossypol, quinoline 3-sulfonamides, N-hydroxyindole-based inhibitors, and FX11. In some embodiments, the amount of lactate dehydrogenase inhibitor in the fermentation medium is between 1-500 mM, 1-400 mM, 1-300 mM, 1-250 mM, between 1-200 mM, 1-175 mM, 1-150 mM, 1-100 mM, 1-50 mM, 1-25 mM, 25-500 mM, 25-400 mM, 25-300 mM, 25-250 mM, 25-200 mM, 25-175 mM, 25-125M, 25-100 mM, 25-75 mM, 25-50 mM, 50-500 mM, 50-400 mM, 50-300 mM, 50-250 mM, 50-200 mM, 50-175 mM, 50-150 mM, 50-125 mM, 50-100 mM, 50-75 mM, 75-500 mM, 75-400 mM, 75-300 mM, 75-250 mM, 75-200 mM, 75-175 mM, 75-150 mM, 75-125 mM, 75-100 mM, 100-500 mM, 100-400 mM, 100-300 mM, 100-250 mM, 100-200 mM, 100-150 mM, 100-125 mM, and 100-500 mM.

In some embodiments, the Bos taurus cells are grown in a suspension culture system. In some embodiments, the cells are grown in a batch, fed-batch, semi continuous (fill and draw) or perfusion culture system or some combination thereof. When grown in suspension culture, the suspension culture can be performed in a vessel (fermentation tank, bioreactor)) of a desired size. The vessel is a size that is suitable for growth of cells without unacceptable rupture of the cells. In some embodiments, the suspension culture system can be performed in vessel that is at least 25 liters (L), 50 L, 100 L, 200 L, 250 L, 350 L, 500 L, 1000 L, 2,500 L, 5,000 L, 10,000 L, 25,000 L, 50,000 L, 100,000 L, 200,000 L, 250,000 L, or 500,000 L. For smaller suspension cultures, the cultivation of the cells can be performed in a flask that is least 125 mL, 250 mL, 500 mL, 1 L, 1.5 L, 2 L, 2.5 L, 3 L, 5 L, 10 L, or larger.

In some embodiments, the cell density of the suspension culture is between 0.25×10⁶ cells.ml, 0.5×10⁶ cells/ml and 1.0×10⁶ cells/ml, between 1.0×10⁶ cells/ml and 2.0×10⁶ cells/ml, between 2.0×10⁶ cells/ml and 3.0×10⁶ cells/ml, between 3.0×10⁶ cells/ml and 4.0×10⁶ cells/ml, between 4.0×10⁶ cells/ml and 5.0×10⁶ cells/ml, between 5.0×10⁶ cells/ml and 6.0×10⁶ cells/ml, between 6.0×10⁶ cells/ml and 7.0×10⁶ cells/ml, between 7.0×10⁶ cells/ml and 8.0×10⁶ cells/ml, between 8.0×10⁶ cells/ml and 9.0×10⁶ cells/ml, between 9.0×10⁶ cells/ml and 10×10⁶ cells/ml, between 10×10⁶ cells/ml and 15.0×10⁶ cells/ml, between 15×10⁶ cells/ml and 20×10⁶ cells/ml, between 20×10⁶ cells/ml and 25×10⁶ cells/ml, between 25×10⁶ cells/ml and 30×10⁶ cells/ml, between 30×10⁶ cells/ml and 35×10⁶ cells/ml, between 35×10⁶ cells/ml and 40×10⁶ cells/ml, between 40×10⁶ cells/ml and 45×10⁶ cells/ml, between 45×10⁶ cells/ml and 50×10⁶ cells/ml, between 50×10⁶ cells/ml and 55×10⁶ cells/ml, between 55×10⁶ cells/ml and 60×10⁶ cells/ml, between 60×10⁶ cells/ml and 65×10⁶ cells/ml, between 70×10⁶ cells/ml and 75×10⁶ cells/ml, between 75×10⁶ cells/ml and 80×10⁶ cells/ml, between 85×10⁶ cells/ml and 90×10⁶ cells/ml, between 90×10⁶ cells/ml and 95×10⁶ cells/ml, between 95×10⁶ cells/ml and 100×10⁶ cells/ml, between 100×10⁶ cells/ml and 125×10⁶ cells/ml, or between 125×10⁶ cells/ml and 150×10⁶ cells/ml.

In some embodiments, the Bos taurus cells are grown while embedded in scaffolds or attached to scaffolding materials. In some embodiments, the Bos taurus cells are differentiated or proliferated in a bioreactor and/or on a scaffold. In some embodiments, the scaffold comprises at least one or more of a microcarrier, an organoid and/or vascularized culture, self-assembling co-culture, a monolayer, hydrogel scaffold, decellularized animal product, such as decellularized meat, decellularized connective tissue, decellularized skin, decellularized offal, or other decellularized animal byproducts, and/or an edible matrix. In some embodiments, the scaffold comprises at least one of plastic and/or glass or other material. In some embodiments, the scaffold comprises natural-based (biological) polymers chitin, alginate, chondroitin sulfate, carrageenan, gellan gum, hyaluronic acid, cellulose, collagen, gelatin, and/or elastin. In some embodiments, the scaffold comprises a protein or a polypeptide, or a modified protein or modified polypeptide. The unmodified protein or polypeptide or modified protein or polypeptide comprises proteins or polypeptides isolated from plants or other organisms. Exemplary plant protein isolates or plant protein concentrates comprise pulse protein, vetch protein, grain protein, nut protein, macroalgal protein, microalgal protein, and other plant proteins. Pulse protein can be obtained from dry beans, lentils, mung beans, faba beans, dry peas, chickpeas, cowpeas, bambara beans, pigeon peas, lupins, vetches, adzuki, common beans, fenugreek, long beans, lima beans, runner beans, or tepary beans, soybeans, or mucuna beans. Vetch protein can be obtained from the genus Vicia. Grain protein can be obtained from wheat, rice, teff, oat, corn, barley, sorghum, rye, millet, triticale, amaranth, buckwheat, quinoa and other grains. Nut protein can be obtained from almond, cashew, pecan, peanut, walnut, macadamia, hazelnut, pistachio, brazil, chestnut, kola nut, sunflower seeds, pumpkin seeds, flax seeds, cacao, pine nut, ginkgo, and other nuts. Proteins obtained from animal source can also be used as scaffolds, including milk proteins, whey, casein, egg protein, and other animal proteins. In some embodiments, the self-assembling co-cultures comprise spheroids and/or aggregates. In some embodiments, the monolayer is with or without an extracellular matrix. In some embodiments, the hydrogel scaffolds comprise at least one of hyaluronic acid, alginate and/or polyethylene glycol. In some embodiments, the edible matrix comprises decellularized plant tissue. Vegetable and animal protein, both modified or unmodified, can be extruded in an extrusion machine to prepare an extrudate that can be used as a scaffold for adherent cell culture of Bos taurus cells. Cultivated animal cells or cells isolated from the tissue of an animal, for example a cultivated Bos taurus cell as disclosed herein or cells isolated from the meat of a cow can be processed through an extrusion machine to make an extrudate, which extrudate can be used as a scaffold for cultivation of Bos taurus cells.

In some embodiments, either primary or secondary Bos taurus cells are modified or grown as in any of the preceding paragraphs.

### Recovery of Cells

The cells can be recovered by any technique apparent to those of skill. In some embodiments the cells are separated from the growth media or are removed from a bioreactor or a scaffold. In certain embodiments, the Bos taurus cells are separated by centrifugation, a mechanical/filter press, filtration, flocculation or coagulation or gravity settling or drying or some combination thereof. In certain embodiments, the filtration method comprises tangential flow filtration, vacuum filtration, rotary vacuum filtration and similar methods. In certain embodiments the drying can be accomplished by flash drying, bed drying, tray drying and/or fluidized bed drying and similar methods. In certain embodiments, the cells are separated enzymatically. In certain embodiments, the cells are separated mechanically.

### Cell Safety

In some embodiments, the population of Bos taurus cells is substantially pure.

In some embodiments, tests are administered at one or more steps of cell culturing to determine whether the Bos taurus cells are substantially pure.

In some embodiments, the Bos taurus cells are tested for the presence or absence of bacteria. In certain embodiments, the types of bacteria tested include, but are not limited to: Salmonella enteritidis, Staphylococcus aureus, Campylobacter jejunim, Listeria monocytogenes, Fecal streptococcus, Mycoplasma genus, Mycoplasma pulmonis, Coliforms, and Escherichia coli.

In some embodiments, components of the cell media, such as Fetal Bovine Serum, are tested for the presence or absence of viruses. In certain embodiments, the viruses include, but are not limited to: Bluetongue, Bovine Adenovirus, Bovine Parvovirus, Bovine Respiratory Syncytial Virus, Bovine Viral Diarrhea Virus, Rabies, Reovirus, Adeno-associated virus, BK virus, Epstein-Barr virus, Hepatitis A virus, Hepatitis B virus, Hepatitis C virus, Herpes Simplex 1, Herpes Simplex 2 , Herpes virus type 6, Herpes virus type 7, Herpes virus type 8, HIV1, HIV-2, HPV-16, HPV 18, Human cytomegalovirus, Human Foamy virus, Human T-lymphotropic virus, John Cunningham virus, and Parvovirus B19.

In some embodiments, the tests are conducted for the presence or absence of yeast and/or molds.

In some embodiments, the tests are for metal concentrations by mass spectrometry, for example inductively coupled plasma mass spectrometry (ICP-MS). In certain embodiments, metals tested include, but are not limited to: arsenic, lead, mercury, cadmium, and chromium.

In some embodiments, the tests are for hormones produced in the culture. In certain embodiments, the hormones include, but are not limited: to 17β-estradiol, testosterone, progesterone, zeranol, melengesterol acetate, trenbolone acetate, megestrol acetate, melengesterol acetate, chlormadinone acetate, dienestrol, diethylstilbestrol, hexestrol, taleranol, zearalanone, and zeranol.

In some embodiments, the tests are in keeping with the current good manufacturing process as detailed by the United States Food and Drug Administration.

### Phenotyping, Process Monitoring and Data Analysis

In some embodiments, the cells are monitored by any technique known to a person of skill in the art. In some embodiments, differentiation is measured and/or confirmed using transcriptional markers of differentiation after total RNA extraction using RT-qPCR and then comparing levels of transcribed genes of interest to reference, e.g., housekeeping genes.

### Food Composition

In certain embodiments provided herein are food compositions or food products comprising Bos taurus cells that are cultivated in vitro. In some embodiments, the cells are combined with other substances or ingredients to make a composition that is an edible food product composition. In certain embodiments, the Bos taurus cells are used alone to make a composition that is a food product composition. In certain embodiments, the food product composition is a product that resembles: nuggets, tenders bites, steak, roast, ground meat, hamburger patties, sausage, or feed stock.

In some embodiments, the recovered Bos taurus cells are prepared into a composition with other ingredients. In certain embodiments, the composition comprises cell paste, mung bean, mung bean protein, fat, and/or water.

In certain embodiments, the food composition or food product has a wet cell paste content of at least 100%, 90%, 80%, 75%, 70%, 65%, 60%, 50%, 40%, 35%, 25%, 15%, 10%, 5% or 1% by weight. In certain embodiments, the food composition or food product has a wet cell paste content by weight of between 10%-20%, 20%-30%, 30%-40%, 40%-50%, 60%-70%, 80%-90%, or 90%-100%. In certain embodiments, the composition comprises a pulse protein content by weight of at least 75%, 70%, 60%, 50%, 40%, 30%, 25%, 20%, or 15% by weight. In certain embodiments, the food composition or food product has a pulse protein content by weight of between 10%-20%, 20%-30%, 30%-40%, 40%- 50%, 60%-70%, 80%-90%, or 90%-95%. In certain embodiments, the food composition or food product comprises a fat content of at least 50%, 40%, 30%, 25%, 20%, 15%, 10%, 5%, or 1% by weight. In certain embodiments, the food composition or food product has a fat content by weight of between 10%-20%, 20%-30%, 30%-40%, 40%- 50%, 60%-70%, 80%-90%, or 90%-95%. In certain embodiments, the food composition or food product comprises a water content of at least 50%, 40%, 30%, 25%, 20%, 15%, 10% or 5% by weight. In certain embodiments, the food composition or food product has a water content by weight of between 10%-20%, 20%-30%, 30%-40%, 40%-50%, 60%-70%, 80%-90%, or 90-95%. In certain embodiments, the food composition or food product comprises a wet cell paste content of between 2%-5%, 5%-10%, 10%-15%, 15%-20%, 20%-25%, 25%-30%, 30%-35%, 35%-40%, 40%-45%, 45%-50%, 50%-55%, 55%-60%, 65%-70%, 70%-75%, 75%-80%, 80%-85%, 85%-90%, or 90%-95%.

In some embodiments, the composition comprises a peptide cross-linking enzyme, for example, transglutaminase content between 0.0001-0.0125%.

In certain embodiments, the food composition or food product comprises a dry cell weight content of at least of 1% by weight. In certain embodiments, the food composition or food product comprises a dry cell weight content of at least of 5% by weight. In certain embodiments, the food composition or food product comprises a dry cell weight content of at least of 10% by weight. In certain embodiments, the food composition or food product comprises a dry cell weight content of at least of 15% by weight. In certain embodiments, the food composition or food product comprises a dry cell weight content of at least of 20% by weight. In certain embodiments, the food composition or food product comprises a dry cell weight content of at least of 25% by weight. In certain embodiments, the composition or food product comprises a dry cell weight of at least of 30% by weight. In certain embodiments, the composition or food product comprises a dry cell weight of at least of 35% by weight. In certain embodiments, the composition or food product comprises a dry cell weight of at least of 40% by weight. In certain embodiments, the composition or food product comprises a dry cell weight of at least of 45% by weight. In certain embodiments, the composition or food product comprises a dry cell weight of at least of 50% by weight. In certain embodiments, the composition or food product comprises a dry cell weight of at least of 55% by weight. In certain embodiments, the composition or food product comprises a dry cell weight of at least of 60% by weight. In certain embodiments, the composition or food product comprises a dry cell weight of at least of 65% by weight. In certain embodiments, the composition or food product comprises a dry cell weight of at least of 70% by weight. In certain embodiments, the composition or food product comprises a dry cell weight of at least of 75% by weight. In certain embodiments, the composition or food product comprises a dry cell weight of at least of 80% by weight. In certain embodiments, the composition or food product comprises a dry cell weight of at least of 85% by weight. In certain embodiments, the composition or food product comprises a dry cell weight of at least of 90% by weight. In certain embodiments, the composition or food product comprises a dry cell weight of at least of 95% by weight. In certain embodiments, the composition or food product comprises a dry cell weight of at least of 97% by weight. In certain embodiments, the composition or food product comprises a dry cell weight of at least of 98% by weight. In certain embodiments, the composition or food product comprises a dry cell weight of at least of 99% by weight. In certain embodiments, the composition or food product comprises a dry cell weight of at least of 100% by weight. In certain embodiments, the food composition or food product comprises a dry cell weight content of between 2%-5%, 5%-10%, 10%-15%, 15%-20%, 20%-25%, 25%-30%, 30%-35%, 35%-40%, 40%-45%, 45%-50%, 50%-55%, 55%-60%, 65%-70%, 70%-75%, 75%-80%, 80%-85%, 85%-90%, or 90%-95%,

In certain embodiments, the food composition or food product comprises a pulse protein content of at least 2%, 5%, 10%, 15%, 20%, 25%, 30%, 35%, 40%, 45%, 50%, 55%, 60%, 65%, 70%, 75%, 80%, 85%, 90% or 95% by weight. In certain embodiments, the food composition or food product comprises a pulse protein content of between 2%-5%, 5%-10%, 10%-15%, 15%-20%, 20%-25%, 25%-30%, 30%-35%, 35%-40%, 40%-45%, 45%-50%, 50%-55%, 55%-60%, 65%-70%, 70%-75%, 75%-80%, 80%-85%, 85%-90%, or 90%-95%, In some embodiments, the pulse protein is a mung bean protein.

In certain embodiments, the food composition or food product comprises, a fat content of at least 1% by weight, a fat content of at least 2% by weight, a fat content of at least 5% by weight, a fat content of at least 7.5% by weight, or a fat content of at least 10% by weight. In certain embodiments, the food composition or food product comprises a fat content of at least 15% by weight. In certain embodiments, the food composition or food product comprises a fat content of at least 20% by weight. In certain embodiments, the food composition or food product comprises a fat content of at least 25% by weight. In certain embodiments, the food composition or food product comprises a fat content of at least 27% by weight. In certain embodiments, the food composition or food product comprises a fat content of at least 30% by weight. In certain embodiments, the food composition or food product comprises a fat content of at least 35% by weight. In certain embodiments, the food composition or food product comprises a fat content of at least 40% by weight. In certain embodiments, the food composition or food product comprises a fat content of at least 45% by weight. In certain embodiments, the food composition or food product comprises a fat content of at least 50% by weight. In certain embodiments, the food composition or food product comprises a fat content of at least 55% by weight. In certain embodiments, the food composition or food product comprises a fat content of at least 60% by weight. In certain embodiments, the food composition or food product comprises a fat content of at least 65% by weight. In certain embodiments, the food composition or food product comprises a fat content of at least 70% by weight. In certain embodiments, the food composition or food product comprises a fat content of at least 75% by weight. In certain embodiments, the food composition or food product comprises a fat content of at least 80% by weight. In certain embodiments, the food composition or food product comprises a fat content of at least 85% by weight. In certain embodiments, the food composition or food product comprises a fat content of at least 90% by weight. In some embodiments, that food composition or food product comprises a fat content of between 1%-5%, between 5%-10%, between 10%-15%, between 15%-20%, between 20%-25%, between 25%-30%, between 30%-35%, between 35%-40%, between 45%-50%, between 50%-55%, between 55%-60%, between 60%-65%, between 65%-70%, between 70%-75%, between 75%-80%, between 80%-85%, between 85%-90%, or between 90%-95%.

In certain embodiments, the food composition or food product comprises a water content of at least 5% by weight. In certain embodiments, the food composition or food product comprises a water content of at least 10% by weight. In certain embodiments, the food composition or food product comprises a water to an amount of 15% by weight. In certain embodiments, the food composition or food product comprises a water content of at least 20% by weight. In certain embodiments, the food composition or food product comprises a water content of at least 25% by weight. In certain embodiments, the food composition or food product comprises a water content of at least 30% by weight. In certain embodiments, the food composition or food product comprises a water content of at least 35% by weight. In certain embodiments, the food composition or food product comprises a water content of at least 40% by weight. In certain embodiments, the food composition or food product comprises a water content of at least 45% by weight. In certain embodiments, the food composition or food product comprises a water content to an amount of 50% by weight. In certain embodiments, the food composition or food product comprises a water content to an amount of 55% by weight. In certain embodiments, the food composition or food product comprises a water content to an amount of 60% by weight. In certain embodiments, the food composition or food product comprises a water content to an amount of 65% by weight. In certain embodiments, the food composition or food product comprises a water content to an amount of 70% by weight. In certain embodiments, the food composition or food product comprises a water content to an amount of 75% by weight. In certain embodiments, the food composition or food product comprises a water content to an amount of 80% by weight. In certain embodiments, the food composition or food product comprises a water content to an amount of 85% by weight. In certain embodiments, the food composition or food product comprises a water content to an amount of 90% by weight. In certain embodiments, the food composition or food product comprises a water content to an amount of 95% by weight.

In one embodiment, the food composition or food product comprises a wet cell paste content between 25-75% by weight, a mung bean protein content between 15-45% by weight, a fat content between 10-30% by weight, and a water content between 20-50% by weight.

In certain embodiments, the food composition or food product comprises peptide cross-linking enzyme. Exemplary peptide cross-linking enzymes are selected from the group consisting of transglutaminase, sortase, subtilisin, tyrosinase, laccase, peroxidase, and lysyl oxidase. In certain embodiments, the composition comprises a cross-linking enzyme of between 0.0001%-0.025%, 0.0001%-0.020%, 0.0001%-0.0175%, 0.0001%-0.0150%, 0.0001%-0.0125%, 0.0001%-0.01%, 0.0001%-0.0075%, 0.0001%-0.005%, 0.0001%-0.0025%, 0.0001%-0.002%, 0.0001%-0.0015%, 0.0001%-0.001%, 0.0001%-0.00015% by weight. In certain embodiments, the food composition or food product comprises a transglutaminase content between 0.0001%-0.025%, 0.0001%-0.020%, 0.0001%-0.0175%, 0.0001%-0.0150%, 0.0001%-0.0125%, 0.0001%-0.01%, 0.0001%-0.0075%, 0.0001%-0.005%, 0.0001%-0.0025%, 0.0001%-0.002%, 0.0001%-0.0015%, 0.0001%-0.001%, 0.0001%-0.00015% by weight. Without being bound by theory, the peptide cross-linking enzyme is believed to cross-link the pulse or vetch proteins and the peptide cross-linking enzyme is believed to cross-link the pulse or vetch proteins to the Bos taurus cells.

In one embodiment, the food composition or food product comprises 0.0001% to 0.0125% transglutaminase, and exhibits reduced or significantly reduced lipoxygenase activity or other enzymes which oxidize lipids, as expressed on a volumetric basis relative to cell paste without the transglutaminase. More preferably, the food composition or food product is essentially free of lipoxygenase or enzymes that can oxidize lipids. In some embodiments, a 5%, 10%, 15%, 20%, 25%, 30%, 35%, 40%, 45%, 50%, 55%, 60%, 65%, 70%, 75%, or 80% reduction in oxidative enzymatic activity relative to a composition is observed. Lipoxygenases catalyze the oxidation of lipids that contribute to the formation of compounds that impart undesirable flavors to compositions.

In some embodiments, mung bean protein is replaced by plant-based protein comprising protein from garbanzo, fava beans, yellow pea, sweet brown rice, rye, golden lentil, chana dal, soybean, adzuki, sorghum, sprouted green lentil, du pung style lentil, and/or white lima bean.

In some embodiments, the addition of additional edible ingredients can be used to prepare the food composition of food product. Edible food ingredients comprise texture modifying ingredients such as starches, modified starches, gums and other hydrocolloids. Other food ingredients comprise pH regulators, anti-caking agents, colors, emulsifiers, flavors, flavor enhancers, foaming agents, anti-foaming agents, humectants, sweeteners, and other edible ingredients.

In certain embodiments, the methods and food composition or food product comprise an effective amount of an added preservative in combination with the food combination.

Preservatives prevent food spoilage from bacteria, molds, fungi, or yeast (antimicrobials); slow or prevent changes in color, flavor, or texture and delay rancidity (antioxidants); maintain freshness. In certain embodiments, the preservative is one or more of the following: ascorbic acid, citric acid, sodium benzoate, calcium propionate, sodium erythorbate, sodium nitrite, calcium sorbate, potassium sorbate, BHA, BHT, EDTA, tocopherols (Vitamin E) and antioxidants, which prevent fats and oils and the foods containing them from becoming rancid or developing an off-flavor.

### Food Process

In some embodiments, provided herein are processes for making a food product that comprises combining pulse protein, Bos taurus cell paste and a phosphate into water and heating up the mixture in three steps. In certain embodiments, the processes comprise adding phosphate to water thereby conditioning the water to prepare conditioned water. In certain embodiments, pulse protein is added to the conditioned water in order to hydrate the pulse protein to prepare hydrated plant protein. In some embodiments, cell paste is added to the hydrated plant protein (conditioned water to which a plant protein has been added) to produce a cell protein mixture. In some embodiments, the plant protein is a pulse protein. In some embodiments, the pulse protein is a mung bean protein.

In some embodiments, the phosphate is selected from the group consisting of disodium phosphate (DSP), sodium hexametaphosphate (SHMP), tetrasodium pyrophosphate (TSPP). In one particular embodiment, the phosphate added to the water is DSP. In some embodiments, the amount of DSP added to the water is at least or about 0.01%, 0.02%, 0.03%, 0.04%, 0.05%, 0.06%, 0.07%, 0.08%, 0.09%, 0.1%, 0.11%, 0.12%, 0.13%, 0.14%, 0.15%, or greater than 0.15%.

In some embodiments, the process comprises undergo three heating steps. In some embodiments, the first heating step comprises heating the cell and protein mixture to a temperature between 40-65° C., wherein seasoning is added. In some embodiments, the second step comprises maintaining the cell and protein mixture at temperature between 40-65° C. for at least 10 minutes, wherein a peptide cross-linking enzyme such as transglutaminase is added. In some embodiments, the third heating step comprises raising the temperature of the cell and protein mixture to a temperature between 60-85° C., where oil is added to the water. In some embodiments, the process comprises a fourth step of lowering the temperature to a temperature between 5-15° C. to prepare a pre-cooking product.

In some embodiments, the seasonings are added to the first step, second step, third step or the fourth step. In some embodiments the seasonings include but are not limited to salt, sugar, paprika, onion powder, garlic powder, black pepper, white pepper, and natural chicken flavor (Vegan).

In some embodiments, the oil (fat) added is to the first step, second step, third step or the fourth step to prepare the pre-cooking product. The oil is selected from the group comprising vegetable oil, peanut oil, canola oil, coconut oil, olive oil, corn oil, soybean oil, sunflower oil, margarine, vegetable shortening, animal oil, butter, tallow, lard, margarine, or an edible oil.

In some embodiments, the pre-cooking product can be consumed without additional preparation or cooking, or the pre-cooking product can be cooked further, using well-known cooking techniques.

In some embodiments, the processes comprise preparing the food product by placement into cooking molds. In some embodiments, the processes comprise applying a vacuum to the cooking molds effectively changing the density and texture of the food product that contains Bos taurus cells cultivated in vitro.

In some embodiments, the food product is breaded.

In some embodiments, the food product is steamed, boiled, sautéed, fried, baked, grilled, broiled, microwaved, dehydrated, cooked by sous vide, pressure cooked, or frozen or any combination thereof.

### Plant Protein Isolation

Plant proteins can be prepared or obtained by any technique or from any source apparent to those of skill. This application references and incorporates the methods for processing plant protein to produce plant protein concentrate and/or plant protein concentrate from US Publication No.: WO2013/067453, US 2017/0238590 A1, WO2017/143298, WO2017/143301, and U.S. 62/981,890 in their entirety.

Provided herein are methods for producing a plant protein isolate or plant protein concentrate having high functionality for a broad range of food applications. In some embodiments, the methods for producing the isolate comprise one or more steps selected from:
(a) extracting one or more or plant protein proteins from a plant protein source in an aqueous solution;
(b) purifying protein from the extract using at least one of two methods:
   (i) precipitating protein from the extract at a pH near the isoelectric point of a globulin-rich fraction, for example a pH between about 5.0-6.0; and/or
   (ii) fractionating and concentrating protein from the extract using filtration methods such as microfiltration, ultrafiltration or chromatography;
(c) recovering purified protein isolate.

In particular embodiments, the plant protein isolate is produced using a series of mechanical processes, with the only chemicals used being pH adjusting agents, such as sodium hydroxide and citric acid, and optionally ethylenediaminetetraacetic acid (EDTA) to prevent lipid oxidation activities affecting the flavor of the isolate.

Although the plant protein isolates or plant protein concentrates provided herein may be prepared from any suitable source of plant protein, where the starting material is whole plant material such as whole mung bean, whole adzuki bean, pea or other plant material, a first step of the methods provided herein typically comprises dehulling the raw source material. In some such embodiments, raw beans are de-hulled in one or more steps of pitting, soaking, and drying to remove the seed coat (husk) and pericarp (bran). The de-hulled mung beans are then milled to produce flour with a well-defined particle distribution size. In some embodiments, the mean particle distribution size is less than 1000, 900, 800, 700, 600, 500, 400, 300, 200 or 100 μm. In a particular embodiment, the particle distribution size is less than 300 μm to increase the rate and yield of protein during the extraction step. The types of mills employed include but are not limited to one or a combination of a hammer, pin, knife, burr, and air classifying mills.

When feasible, air classification of the resultant flour may expedite the protein extraction process and enhance efficiency of the totality of the process. The method employed is to ensure the beans are milled to a particle size that is typically less than 45 μm, utilizing a fine-grinding mill, such as an air classifying mill. The resultant flour is then passed through an air classifier, which separates the flour into both a coarse and fine fraction. The act of passing the flour through the air classifier is intended to concentrate the majority of the available protein in the flour into a smaller portion of the total mass of the flour. Typical fine fraction (high-protein) yields are 5-50%. The fine fraction tends to be of a particle size of less than 20 μm; however, this may be influenced by growing season and region of the original bean. The high-protein fraction typically contains 150-220% of the protein in the original sample. The resultant starch-rich byproduct stream also becomes value added, and of viable, saleable interest as well.

In preferred embodiments, the methods to purify plant protein isolate or plant protein concentrate comprise an extraction step. In some embodiments of the extraction step, an intermediate starting material, for example, bean flour, is mixed with aqueous solution to form a slurry. In some embodiments, the aqueous solution is water, for example soft water. The aqueous extraction includes creating an aqueous solution comprising one part of the source of the plant protein (e.g., flour) to about, for example, 2 to 15 parts aqueous extraction solution. In other embodiments, 5 to 10 volumes of aqueous extraction solution is used per one part of the source of the plant protein. Additional useful ratios of aqueous extraction solution to flour include 1:1, 2:1, 4:1, 6:1, 7:1, 8:1, 9:1, 10:1, 11:1, 12:1, 13:1, 14:1, 15:1 or alternatively 1:2, 1:3, 1:4, 1:5, 1:6, 1:7, 1:8, 1:9, 1:10, 1:11, 1:12, 1:13, 1:14, 1:15.

Preferably, the aqueous extraction is performed at a desired temperature, for example, about 2-50° C. in a chilled mix tank to form the slurry. In some embodiments, the mixing is performed under moderate to high shear. In some embodiments, a food-grade de-foaming agent (e.g., KFO 402 Polyglycol) is added to the slurry to reduce foaming during the mixing process. In other embodiments, a de-foaming agent is not utilized during extraction.

In some embodiments, sequential extraction with multiple stages is performed to improve the extraction.

In some embodiments, the sequential extraction is performed either in batch mode or continuous mode

In some embodiments the sequential extraction is performed in current or counter current mode.

The pH of the slurry is adjusted with a food-grade 50% sodium hydroxide solution to reach the desired extraction pH for solubilization of the target protein into the aqueous solution. In some embodiments, the extraction is performed at a pH between about 5-10.0. In other embodiments, the extraction is performed at neutral or near neutral pH. In some embodiments, the extraction is performed at a pH of about pH 5.0-pH 9, pH 6.0-pH 8.5 or more preferably pH 6.5-pH 8. In a particular embodiment, the extraction is performed at a pH of about 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In a particular embodiment, the extraction is performed at a pH of about 7.0.

Following extraction, the solubilized protein extract is separated from the slurry, for example, in a solid/liquid separation unit, consisting of a decanter and a disc-stack centrifuge. The extract is centrifuged at a low temperature, preferably between 3-10° C. The extract is collected, and the pellet is resuspended, preferably in 3:1 water-to-flour. The pH is adjusted again and centrifuged. Both extracts are combined and filtered through using a Nylon mesh.

Optionally, the protein extract is subjected to a carbon adsorption step to remove non-protein, off-flavor components, and additional fibrous solids from the protein extraction. This carbon adsorption step leads to a clarified protein extract. In one embodiment of a carbon adsorption step, the protein extract is then sent through a food-grade granular charcoal-filled annular basket column (<5% w/w charcoal-to-protein extract ratio) at 4 to 8° C.

In some embodiments, following extraction and optionally carbon adsorption, the clarified protein extract is acidified with a food-safe acidic solution to reach its isoelectric point under chilled conditions (e.g., 2 to 8° C.). Under this condition, the target protein precipitates and becomes separable from the aqueous solution. In some embodiments, the pH of the aqueous solution is adjusted to approximately the isoelectric point of at least one of the one or more globulin-type proteins in the protein-rich fraction, for example, mung bean 8S/beta conglycinin. In some embodiments, the pH is adjusted from an aqueous solution comprising the protein extract which has an initial pH of about 5.0-10.0 prior to the adjusting step. In some embodiments, the pH is adjusted to about 5.0 to 6.5. In some embodiments, the pH is adjusted to about 5.2-6.5, 5.3 to 6.5, 5.4 to 6.5, 5.5 to 6.5, or 5.6 to 6.5. In some embodiments, the pH is adjusted to about 5.2-6.0, 5.3 to 6.0, 5.4 to 6.0, 5.5 to 6.0, or 5.6 to 6.0. In certain embodiments, the pH is adjusted to about pH 5.4-5.8. In some embodiments, the pH is adjusted to about 5.2, 5.3, 5.4, 5.5, 5.6, 5.7, 5.8, 5.9, 6.0, 6.1, or 6.2.

In a preferred embodiment of the methods provided herein, for mung bean protein purification, the pH is adjusted, and precipitation of desired mung bean proteins is achieved, to a range of about pH 5.6 to pH 6.0. Without being bound by theory, it is believed that isoelectric precipitation at a range of about pH 5.6 to pH 6.0 yields a superior mung bean protein isolate, with respect to one or more qualities selected from protein yield, protein purity, reduced retention of small molecular weight non-protein species (including mono and disaccharides), reduced retention of oils and lipids, structure building properties such as high gel strength and gel elasticity, superior sensory properties, and selective enrichment of highly functional 8S globulin/beta conglycinin proteins. These unexpectedly superior features of mung bean protein isolates or mung bean protein concentrates prepared by the methods provided herein are described, for example, in Examples 6 and 8 of US Publication No.: US 2017/0238590 A1. As demonstrated by the results described in Example 6 of US2017/0238590 A1, mung bean protein isolates that underwent acid precipitations at a pH range of about pH 5.6 to pH 6.0 demonstrated superior qualities with respect to protein recovery (in comparison to recovery of small molecules), gelation onset temperature, gel strength, gel elasticity, and sensory properties, in comparison to mung bean protein isolates that underwent acid precipitations at a pH below pH 5.6. Mung bean protein isolates that underwent acid precipitations at a pH range of about pH 5.2 to pH 5.8 also demonstrated substantially lower lipid retention when compared to mung bean protein isolates that underwent acid precipitations outside this range.

Suitable food-grade acids to induce protein precipitation include but are not limited to malic, lactic, hydrochloric acid, and citric acid. In a particular embodiment, the precipitation is performed with a 20% food-grade citric acid solution. In other embodiments, the precipitation is performed with a 40% food-grade citric acid solution.

In some embodiments, in addition to the pH adjustment, EDTA, for example, 2 mM of food-grade EDTA, is added to the precipitation solution to inhibit lipid oxidation in order to produce off-flavor compounds.

In alternative embodiments, the precipitation step comprises isoelectric precipitation at pH 5.6 combined with cryo-precipitation (at 1-4° C.), wherein the pH is adjusted to 5.4-5.8.

In another alternative embodiment, low ionic strength precipitation at high flow rates is combined with cryo-precipitation (at 1-4° C.). In some such embodiments, rapid dilution of the filtrate is performed in cold (1-4° C.) 0.3% NaCl at a ratio of 1 volume of supernatant to 3 volumes of cold 0.3% NaCl. Additional resuspension and homogenization steps ensure production of desired protein isolates.

In some embodiments, the precipitated protein slurry is then removed from the pH-adjusted aqueous solution and sent to a solid/liquid separation unit (for example, a one disc-stack centrifuge). In some embodiments of the methods, the separation occurs with the addition of 0.3% (w/w) food-grade sodium chloride, and a protein curd is recovered in the heavy phase. In preferred embodiments the protein curd is washed with 4 volumes of soft water under chilled conditions (2 to 8° C.), removing final residual impurities such as fibrous solids, salts, and carbohydrates.

In some embodiments of the methods, filtration is used as an alternative, or an addition to, acid precipitation. Without being bound by theory, it is believed that while acid precipitation of the protein aids to remove small molecules, alternative methods such as ultra-filtration (UF) are employed to avoid precipitation/protein aggregation events. Thus, in some embodiments, purifying the protein-rich fraction to obtain the mung bean protein isolate or mung bean protein concentrate comprises performing a filtration, microfiltration or ultrafiltration procedure utilizing at least one selective membrane.

The ultrafiltration process utilizes at least one semi-permeable selective membrane that separates a retentate fraction (containing materials that do not pass through the membrane) from a permeate fraction (containing materials that do pass through the membrane). The semi-permeable membrane separates materials (e.g., proteins and other components) based on molecular size. For example, the semi-permeable membrane used in the ultrafiltration processes of the present methods may exclude molecules (i.e., these molecules are retained in the retentate fraction) having a molecular size of 10 kDa or larger. In some embodiments, the semi-permeable membrane may exclude molecules (e.g., pulse proteins) having a molecular size of 25 kDa or larger. In some embodiments, the semi-permeable membrane excludes molecules having a molecular size of 50 kDa or larger. In various embodiments, the semi-permeable membrane used in the ultrafiltration process of the methods discussed herein excludes molecules (e.g., pulse proteins) having a molecular size greater than 5 kDa, 10 kDa, 15 kDa, 20 kDa, 25 kDa, 30 kDa, 35 kDa, 40, kDa, 45 kDa, 50 kDa, 55 kDa, 60 kDa, 65 kDa, 70 kDa, 75 kDa, 80 kDa, 85 kDa, 90 kDa, or 95 kDa. For example, a 10 kDa membrane allows molecules, including pulse proteins, smaller than 10 kDa in size to pass through the membrane into the permeate fraction, while molecules, including pulse proteins, equal to or larger than 10 kDa are retained in the retentate fraction.

In some embodiments, the washed protein curd solution resulting from acid precipitation and separation is pasteurized in a high temperature/short time pasteurization step to kill any pathogenic bacteria present in the solution. In a particular embodiment, pasteurization is performed at 74° C. for 20 to 23 seconds. In particular embodiments where a dry isolate is desired, the pasteurized solution is passed through a spray dryer to remove any residual water content. The typical spray drying conditions include an inlet temperature of 170° C. and an outlet temperature of 70° C. The final dried protein isolate powder typically has less than 5% moisture content. In some embodiments of the methods described herein, the pasteurization is omitted, to maintain broader functionality of the protein isolate.

The following non-limiting methods are provided to further illustrate the embodiments of the invention disclosed herein. It should be appreciated by those of skill in the art that the techniques disclosed in the examples that follow represent approaches that have been found to function well in the practice of several embodiments of the invention, and thus be considered to constitute examples of modes for its practice. However, those of skill in the art should, in light of the present disclosure, appreciate that many changes can be made in the specific embodiments that are disclosed and still obtain a like or similar result without departing from the spirit and the scope of the invention.

### EXAMPLES

### Example 1

### Isolation of Primary Muscle Cells

Isolation of Satellite Cells from Muscle Tissue

Myosatellite cells were isolated from muscle tissue from a BWF animal (black-white face; a cross between Aberdeen Angus and Hereford breeds) within 4 h of slaughter at a farm in Northern California. The tissue was rinsed with Hank's Basic Salt Solution (HBSS) with Ca²⁺ and Mg²⁺ (catalogue number 14025092, Life Technologies) and transferred to a sterile plate. Connective tissue, blood vessels, nerve bundles and adipogenic tissue were removed using sterile forceps and scalpels, and the tissue was cut into small fragments of approximately 2-3 mm³ while maintaining the tissue moist with HBSS.

The minced tissue was transferred to a container and washed with an equal volume of HBSS and the tissue fragments were left to sediment. The wash solution was removed and 3.5 mL of dilution of collagenase solution in HBSS per gram of tissue (Liberase™, catalogue number 05401127001, Roche with a final concentration of 1.0 Wunsch units per mL) was added. Tissue fragments were transferred to a tissue culture incubator with a shaker platform for dissociation for 45 minutes with slow rotation (50 rpm). Digested tissue was vigorously mixed to release single cells from the minced tissue. The cell suspension was passed through a sterile metal sieve to remove larger fragments and the dissociated cells were collected in a flask. The collected fragments were mixed with the same volume of serum containing media (DMEM, catalogue number 11960077, Life Technologies supplemented with 10% FBS, catalogue number 1300-050, Seradigm) to inactivate the dissociation enzymes, vigorously mixed and passed though the sieve as before, pooling it with the dissociated cells. The cell suspension was filtered sequentially through 100 μm and 40 μm cell strainers to eliminate residual large tissue fragments. The dissociated cells were collected by centrifuging the cell suspension at 1000 g at 4° C. for 20 min. The supernatant was carefully removed, the cell pellet was resuspended in 40 mL of serum-containing media and transferred to a 50 mL conical tube for collection by centrifugation at 400 g at 4° C. for 5 min.

Isolated cells from muscle tissue were resuspended in 40 mL of Skeletal Muscle Cell Growth Medium (abbreviated SKGM, catalogue number C-23060, PromoCell, with 1× SupplementMix, catalogue number: C-39365, PromoCell) supplemented with 1× antibiotic/antimycotic mix (catalogue number 15240062, ThermoFisher) and 1:1000 (v/v) Primocin (catalogue number ant-pm-1, InvivoGen) and seeded in two T-175 treated cell culture flasks (catalogue number 83.3912.002, Sarstedt) per 10 g of tissue, previously coated with 10 mL of gelatin solution (EmbryoMax 0.1% gelatin solution in water, catalogue number ES-006-G, Millipore Sigma). Cells were incubated undisturbed for 72 h at 37° C., 5% CO₂. Beef myo-progenitor cells were named as B4M cells.

After the initial incubation, B4M cultures were observed under the microscope and confirmed to have the spread-out, stellate morphology of early progenitor cells grown in adherent conditions (Yablonka-Reuveni and Nameroff 1987). Cell debris was removed by aspiration, and the growing B4M cells were washed gently (to minimize cell detachment) once or twice with 25 mL HBSS per flask, and expansion continued with 25 mL SKGM media with antibiotic/antimycotic mixture per flask until culture became 70-80% confluent.

After incubation for another 48-72 h, B4M cultures reached the desired confluency and were harvested. Cultures in each flask were washed with 10 mL Dulbecco's phosphate-buffered saline (DPBS, catalogue number 14190-144, ThermoFisher) and dissociated by incubation with 5 mL TrypLE Express (catalogue number 12505-010, ThermoFisher) for 5-8 min at 37° C., 5% CO₂. Cultures were observed under the microscope and when cell dissociation from the culture surface was complete, the dissociation reaction was stopped by the addition of equal volume of complete media. The cell suspension was transferred to a 50 mL conical tube, cells were collected by centrifugation at 400 g at room temperature for 5 min.

Primary cells recovered from this first step of expansion were labelled as p0 (passage 0) and were cryopreserved by resuspending the cell pellet in freezing media (SKGM supplemented with 10% (v/v) DMSO, catalogue number D2650-5×10ML, Millipore Sigma) at 1-5×10⁶ cell/mL/vial following standard cryopreservation methods for mammalian cells, creating a parental Research Cell Bank (RCB).

Early cultures of isolated B4M satellite cells were characterized by their proliferation ability, the expression of myogenic markers and their capability to differentiate into cells of more mature myogenic phenotype.

Isolated B4M satellite cells were expanded in gelatin-coated T-75 or T-175 cell culture flasks (catalogue numbers 83.3912.002 or 83.3912.002, Sarstedt). B4M cells were seeded at 2,800-3,000 cell/cm², in 10 mL or 25 mL of SKGM media respectively, and cells were harvested when cultures reached 70-80% confluency, approximately every 3 to 4 days.

Population doubling time (PDT) and Population doubling level (PDL) were calculated according to the following formulae, considering PDL of 0 for cells at p0:

PDT=t*log 10(2)/[(log 10(n/n0)] and PDL=3.32[log 10(n/n0)]
where t=time in culture, n=final cell number and n0=number of cells seeded.

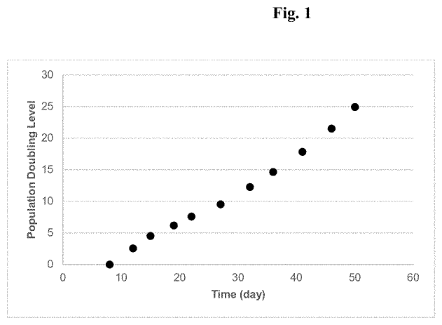

B4M progenitor cells isolated from bovine muscle tissue were able to reach 25 population doublings (FIG. 1) and to proliferate with an average population doubling time of approximately 43 h for 50 days (10 passages since isolation) in culture under the conditions described above.

### Example 2

### Cell Surface Marker Expression

The expression of myogenic (CD56) and stemness (CD29) cell markers were determined in cultures from isolated B4M satellite cells. The surface marker CD56, neural-cell adhesion molecule (NCAM), first identified in the surface of neurons and glia, is also characteristically expressed on the surface of skeletal muscle cells (Verdijk et al., 2014), while CD29 is a marker of mesenchymal stromal cells (Yang et al., 2014) and is also expressed in cardiac and skeletal muscle (Sastry and Horwitz, 1993).

Adherent cultures of isolated B4M satellite cells of Example 1, at different passages, were dissociated into single cell suspensions, washed with DPBS solution and approximately 1×10⁶ cells were incubated with blocking buffer (10% FBS dilution in autoMACS running buffer [catalogue number 130-091-221, Miltenyi]) for 10-20 minutes at room temperature, to block targets for the non-specific binding of antibodies. Cell suspensions containing approximately 250,000 cells were used for each of the staining conditions, including an unstained condition, an isotype control condition (PE/Cy7-IgG2a,k isotype control; clone MOPC-173, catalogue number 400232, BioLegend) and test conditions stained with CD56 (PE/Cy7-CD56 antibody; clone MEM-188; catalogue number 304628, BioLegend) and CD29 (PE/Cy7-CD29 antibody; clone TS2/16, catalogue number 303026, BioLegend), respectively. For each condition, cells were collected by centrifugation at 500 g at room temperature for 5 min, resuspended in 100 μL of antibody dilution (following manufacturer's recommendation) in blocking buffer according to the conditions specified above; and incubated at least 30 minutes, and no longer than 2 h, at 2-8° C. or on ice, protecting the sample from light.

After staining, samples were washed twice with 1 mL of autoMACS running buffer, collecting cells by centrifugation at 500 g at room temperature for 5 min. Following the last wash, cells were resuspended in 200-300 μL of autoMACS running buffer and analyzed in an Accuri C6 system (BD Biosciences, CA).

The majority of the expanded B4M cells from those isolated from bovine muscle tissue expressed CD56 and CD29 markers. The percentage of B4M cells expressing those markers decreased with passage and time in culture; CD56 expression showed a constant decrease, with levels as low as being expressed in 20% of the cells by the end of the short-term proliferation study, while levels of CD29 decreased to around 25% of the cells at passage 5 before increasing to high percentages in later passages. The stemness marker, expressed in a fraction of cells, provided a proliferative advantage as there was an enrichment in the population of cells expressing the CD29 marker in the continued expanded culture of B4M cells isolated from muscle tissue.

Expanded B4M cells from those isolated cells from bovine muscle were tested for the expression of several stemness, hematopoietic, and early, intermediate and late myogenic markers to characterize the gene expression levels of these myosatellite markers.

For RNA isolations, 2-3×10⁶ cells grown as described in Example 1 were collected. Cell pellets were washed with PBS buffer and cell pellets stored at −80° C. until processing. Total RNA was extracted using RNeasy plus Mini Kit (catalogue number 74136, Qiagen) or Quick RNA Miniprep Kit (catalogue number R1055, Zymo Research) according to the manufacturer's instructions, including in column DNase treatment. Total RNA was eluted in RNase-free water and stored at −80° C. Concentration and quality of RNA was determined with a NanoDrop spectrophotometer (Thermo Scientific).

cDNA was produced using High-Capacity cDNA Reverse Transcription Kit (catalogue number 4368814, Thermo Fisher) as per the manufacturer's recommendations, using 1 μg of total RNA in a 25 μL reaction using random primers. Briefly, reactions were incubated at 25° C. for 10 minutes and then at 37° C. for 2 h; the reverse transcriptase was inactivated by heating up to 85° C. for 5 min before cooling down the reaction to 4° C.; cDNA was stored at −20° C.

cDNA samples were diluted 10-fold, and 5 μL of sample were used as the template for gene expression characterization by PCR in a 20 μL volume reaction. Amplifications were performed using DreamTaq™ Hot Start Green PCR Master Mix (catalogue number ferk9021, Thermo Fisher) following the manufacturer's recommendations, using 0.5 μM of each of the primers shown in Table 1.

TABLE 1
Primer sequences used for amplification of
markers for phenotypic analysis

| | | Ampli- | | |
|---|---|---|---|---|
| | Acces- | con | | |
| Primer | sion | Size | Forward | Reverse |
| Set | Number | (bp) | Primer | Primer |
| CD29 | NM_ | 193 | TGTCGAGTGT | AGACTCCAAG |
| | 174368 | | GTGAGTGCAA | GCAGGTCTGA |
| | | | (SEQ ID | (SEQ ID |
| | | | NO: 1) | NO: 2) |
| CD90 | NM_ | 201 | GTGAACCAGA | GGTGGTGAAG |
| | 001034765 | | GCCTTCGTCT | TTGGACAGGT |
| | | | (SEQ ID | (SEQ ID |
| | | | NO: 3) | NO: 4) |
| CD105 | NM_ | 226 | CTGATCCTCA | GACGAAGGAA |
| | 001076397 | | GCGTGAACAA | GATGCTTTGC |
| | | | (SEQ ID | (SEQ ID |
| | | | NO: 5) | NO: 6) |
| PAX3 | NM_ | 77 | AAAAGAGAGA | GTGTTTCGAT |
| | 001206818 | | ACCCCGGCAT | CACAGACCGC |
| | | | (SEQ ID | (SEQ ID |
| | | | NO: 7) | NO: 8) |
| PAX7 | XM_ | 216 | GGGCATGTTT | TCCAGACGGT |
| | 015460690 | | AGCTGGGAGA | TCCCTTTGTC |
| | | | (SEQ ID | (SEQ ID |
| | | | NO: 9) | NO: 10) |
| Myf5 | NM_ | 192 | CTGCTTAGGG | GGAGCTTTTA |
| | 174116 | | AACAGGTGGA | TCCGTGGCAT |
| | | | (SEQ ID | AT (SEQ ID |
| | | | NO: 11) | NO: 12) |
| Mrf4 | NM_ | 282 | GCGAAAGGAG | TGGAATGATC |
| | 002469 | | GAGGCTAAAG | GGAAACACTT |
| | | | AAAATCAACG | GGCCACTG |
| | | | (SEQ ID | (SEQ ID |
| | | | NO: 13) | NO: 14) |
| MyoD | NM_ | 239 | GTCTAGCAAC | GGCCGCTGTA |
| | 001040478 | | CCAAACCAGC | GTCCATCAT |
| | | | (SEQ ID | (SEQ ID |
| | | | NO: 15) | NO: 16) |
| MyoG | NM_ | 162 | GGCGGTGCCC | ACTGTGATGC |
| | 001111325 | | AGTGAAT | TGTCCACGAT |
| | | | (SEQ ID | G (SEQ ID |
| | | | NO: 17) | NO: 18) |
| S100A4 | NM_ | 185 | TCTCTTGCTC | ACGCAGTTTC |
| | 174595 | | CTGACTGCTG | ATCCGTCCTT |
| | | | (SEQ ID | (SEQ ID |
| | | | NO: 19) | NO: 20) |
| CD56 | NM_ | 166 | CAAATACCGA | GGTCCTGAAC |
| | 174399 | | GCGCTCTCCT | ACAAAGTGCG |
| | | | (SEQ ID | (SEQ ID |
| | | | NO: 21) | NO: 22) |
| CD82 | NM_ | 139 | GGGGATGTAC | GCCGTCCGTG |
| | 001097990 | | TTTGCCTTCC | TAGTTGTGA |
| | | | T (SEQ ID | (SEQ ID |
| | | | NO: 23) | NO: 24) |
| PDGFR | NM_ | 168 | CATCTACGTG | CTGTCATAGG |
| A | 001192345 | | CCAGACCCAG | AGGCAGGCAC |
| | | | (SEQ ID | (SEQ ID |
| | | | NO: 25) | NO: 26) |
| CDKN1 | NM_ | 72 | GCAGACCAGC | TGGGGTTAGG |
| A | 001098958 | | ATGACAGATT | GCTTCCTCTT |
| | | | TC (SEQ ID | (SEQ ID |
| | | | NO: 27) | NO: 28) |
| CDKN2 | XM_ | 149 | AGCAGCATGG | CTGCCCATCA |
| A | 010807759 | | AGACCTCGG | TCATCACCTG |
| | | | (SEQ ID | AATC |
| | | | NO: 29) | (SEQ ID |
| | | | | NO: 30) |
| MyHC | NM_ | 116 | AGAGCAGCAA | TGGACTCTTG |
| | 001166227 | | GTGGATGACC | GGCCAACTTG |
| | | | TTGA | AGAT |
| | | | (SEQ ID | (SEQ ID |
| | | | NO: 31) | NO: 32) |
| Desmin | NM_ | 133 | CCTCAAGGAT | GATAGGGAGG |
| | 001081575 | | GAGATGGCCC | TTGATCCGGC |
| | | | (SEQ ID | (SEQ ID |
| | | | NO: 33) | NO: 34) |
| PECAM | NM_ | 123 | ACAGTTGAGG | TGAGAAGGAT |
| | 174571 | | AGCAAGACCG | TCCCGCACAG |
| | | | (SEQ ID | (SEQ ID |
| | | | NO: 35) | NO: 36) |
| CD34 | NM_ | 88 | CAGTCACCTT | TGGACAGAAG |
| | 174009 | | AGTTCCAGCG | AGTTCACGGC |
| | | | T (SEQ ID | (SEQ ID |
| | | | NO: 37) | NO: 38) |
| RPL32 | NM_ | 186 | CAAAATCAAG | CACATCAGCA |
| | 001034783 | | CGGAACTGGC | GCACCTCAAG |
| | | | (SEQ ID | (SEQ ID |
| | | | NO: 39) | NO: 40) |

The Hot Start polymerase was activated at 95° C. for 3 min, followed by 35 cycles of amplification composed of a denaturing step of 95° C. for 10 sec, annealing and extension steps of 55° C. for 15 sec and 72° C. for 30 s respectively, and a final extension of 72° C. for 5 min. A fraction of the PCR mixture (10 μL) was run in a 2% agarose gel (E-gel agarose gels 2%, catalogue numbers G800802 and G601802, Thermo Fisher) and expression of the different markers tested determined by the presence or absence of the corresponding amplicon.

Cultures that were expanded in vitro expressed high levels of the early paired-box/homeobox transcription factors Pax3 and Pax7, as well as high levels of expression of four intermediate muscle-specific transcription factors, Myf5, Mrf4, MyoD and MyoG, that defined a population of muscle progenitor cells (Alonso-Martin et al., 2016). These cells also expressed other myogenic surface markers like CD56 and CD82 (Alexander et al., 2016), suggesting they were committed early myoblasts. The absence of mature myogenic markers, exemplified by Desmin and Myosin Heavy chain (MyHC2), confirmed that isolated and expanded cells were in an early stage of myogenesis differentiation, and they had not reached the myofiber commitment stage at this point (Glaser and Suzuki, 2018). Isolated and expanded cells from bovine muscle comprised a mixture of cells, mainly muscle progenitors as shown by the presence of cell markers expressed by myosatellite cells.

### Example 3

### Myogenic Differention of Myosatellite Cells

As shown in Example 2, the isolated and expanded B4M cells from bovine muscle expressed genes that are characteristic of cells with myogenic potential. To confirm that the isolated B4M cells can indeed differentiate into cells with a more mature muscle phenotype, cultures of adherent B4M cells were exposed to no-serum conditions that cease cellular division and start the terminal differentiation program into mature myogenic cells that eventually leads to the formation of myotubes.

Cultures of early expanded cells (B4M p1) were seeded in 4× wells of a tissue culture-treated 6-well plates at approximately 100,000 cells/well in 2 mL of SKGM growth media. Cells were incubated undisturbed for 72 h at 37° C., 5% CO₂, allowing cultures to reach confluency levels of 80-100%. At this point, growth media was removed, attached cells were washed twice with 2 mL/well DPBS to move any remaining traces of serum from the growth media. Cultures growing in 2 of the wells were used as control conditions, and cultures continued to be incubated in growth media, SKGM, for the entirety of the differentiation protocol period, while the other 2 wells followed the differentiation conditions, where 2 mL/well of Skeletal Muscle Cell Differentiation Media (abbreviated SKDM, catalogue number C-23060, PromoCell, with 1× Cell Differentiation SupplementMix, catalogue number: C-39366, PromoCell) was added to these cultures. Incubation continued as before for an additional 10 days, changing the corresponding media every 3-4 days and allowing cells to follow the differentiation process.

At the end of the differentiation period, cultures were observed under the microscope and cell morphology for control B4M cells (cultured with SKGM media) and differentiated B4M cells (cultured with SKDM media) were documented.

For phenotypic analysis of these cultures, RNA was isolated from the attached cultures. Modification to the protocol disclosed in Example 2 included the washing of the attached cultures with 2 mL/well DPBS and the addition of 350 μL of lysis buffer directly on top of the cultured cells. Lysis of cells was promoted by the placement of the 6-well plate in a shaker for 5 min at room temperature. The lysate was collected with a micropipette, removing all material attached to the culture plate and transferring to the RNA purification column; at this point manufacturer's instructions were followed.

At the end of the differentiation culture period, B4M cells were observed under the microscope and the formation of very thin and elongated cells, corresponding to myoblasts and myocytes, were identified as cells that have committed to a myogenic lineage. In contrast, undifferentiated cells incubated in growth media showed more rounded form. At this point in the differentiation process, multiple cells fused together to form multinucleated cells were observed, demonstrating that the second stage of differentiation that involves the alignment and fusion of myoblasts had occurred.

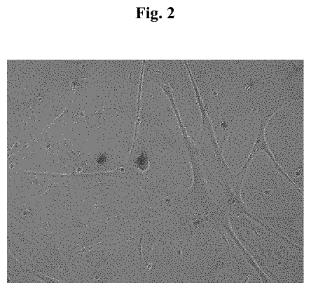

FIG. 2 provides a phase contrast microscope image showing the differentiation of myosatellite cells into myotubes/myofibers. The formation of thin and elongated multinucleated cells shows the fusion of myosatellite cells, characteristic of myoblasts and myocytes.

### Example 4

### Transduction of Myosatellite Cells

Recent developments with human cells have shown that the ectopic expression of telomerase reverse transcriptase, the catalytic subunit of the telomerase enzyme (Nugent 1998; Sealey 2010; Wieser 2008), has led to continued cell replication and generation of immortalized human cell lines (Lee 2004). Here, we demonstrate that expression of telomerase reverse transcriptase creates an immortalized cell line derived from progenitor cells isolated from bovine muscle tissue.

Introduction of genetic material into primary and stem cells is a challenge as the efficiency of this process is low for the most common delivery methods using chemical reagents (e.g., transfection protocols that use lipids) or by physical approaches (e.g., electroporation, magnetic delivery). A more efficient approach is the delivery of the genetic information using a biological entity, like a virus or virus-like particle; and more recently, lentiviral vectors have become widely used for gene delivery.

Telomerase reverse transcriptase (TERT) is the catalytic subunit of telomerase, which together with the telomerase RNA component (TERC) form the telomerase enzyme. Telomerase binds to the telomerase associated protein 1 (TEP1), the heat shock protein 9 (hsp90), p23 and dyskerin to form a holoenzyme complex. While TEP1 is associated with RNA and protein binding activities, and p23 and dyskerin act as molecular chaperons which binds to TERT, the latter one is the main catalytic component of the complex (Holt 1999).

The telomerase complex is responsible for lengthening telomeres, the repeated sequences (TTAGGG in all vertebrates) at the end of the DNA strands in the chromosomes (Blackburn 1991). Telomerase and its associated proteins are highly conserved across species. Telomerase acts to i) maintain proper segregation of chromosomes by preventing the fusion of chromosomes ends and ii) protect the coding DNA regions from the incomplete DNA replication that would lead to progressive loss of chromosomal ends (Blackburn 1991). Telomerase activity is detected in stem cells, germ cells and other cells that divide rapidly, as well as immortalized and tumor cells in vitro and in primary tumor tissues, while low or no activity is detected in somatic cells (Flores 2006, Nussey 2014). Reduced telomerase activity is associated with replicative senescence, the finite cellular replication that leads to cell cycle arrest when telomeres are shorter than a certain length, the Hayflick limit, as firstly suggested by Hayflick (Hayflick 1965).

The amino acid sequence of TERT from Bos taurus (cattle, taxon: 9913) can be accessed at the NCBI database with accession number NP_001039707 (last annotated on 16 Dec. 2019) and with RefSeq (provisional) NM_001046242.1 (Szczotka 2013, Garrels 2012, Zimin 2009). The bovine TERT gene (gene ID: 518884, ensemble ID: ENSBTAT00000012567) is located in chromosome 20 in location NC_037347.1 (71145303.71162377), according to the current assembly for Bos taurus (ARS-UCD1.2-GCF_002263795.1, annotation release 106). This sequence was derived from an animal of the Hereford breed.

The TERT gene is a single copy gene with a single transcription start site, but subject to alternatively splicing regulation with at least 4 splicing variant identified in the NCBI database (XM_024981282.1, XM_024981283.1, XM_024981284.1) showing alternative splicing at the 5′ end of the gene when compared to the annotated full-length TERT mRNA, NM_001046242.1). The ensemble database contains 2 splicing variants (of 3378 and 3261 bp, resulting in protein coding regions of 1125 and 1086 amino acids [aa]) similar to the above and a third one showing an alternative start site (3285 bp and 449 aa) that results in truncated protein containing mainly the catalytic domain. The TERT gene comprises 16 exons and 15 introns within a ˜17 kb body; the coding region is comprised of 3378 nucleotides (NM_001046242.1), resulting in a main protein of 1125 aa in length.

The Exon structure of the TERT gene in B taurus was generated with Splign mapping the coding region NM_001046242 to that of Bos taurus (isolate L1 Dominette 01449 registration number 42190680 breed Hereford chromosome 20, ARS-UCD1.2).

The coding region of btTERT was subcloned from CloneID OBa228014 (GenScript) by GenTarget using the proprietary Eco cloning method. The subcloned insert was verified by sequencing analysis by GenTarget according to the report for service. The translated protein sequence derived from the coding sequence subcloned into the lentivirus transfer plasmid is shown below and it is identical to that of NP_001039707, the telomerase catalytic subunit (EC number 2.7.7.49). The calculated molecular weight of TERT is 124,316 Da.

```
(SEQ ID NO: 41)
      1 MPRAPRCRAV RALLRASYRQ VLPLAAFVRR
          LRPQGHRLVR RGDPAAFRAL VAQCLVCVPW
    61 DAQPPPAAPS FRQVSCLKEL VARVVQRLCE
          RGARNVLAFG FTLLAGARGG PPVAFTTSVR
  121 SYLPNTVTDT LRGSGAWGLL LHRVGDDVLT
          HLLSRCALYL LVPPTCAYQV CGPPLYDLRA
  181 AAAAARRPTR QVGGTRAGFG LPRPASSNGG
          HGEAEGLLEA RAQGARRRRS SARGRLPPAK
  241 RPRRGLEPGR DLEGQVARSP PRVVTPTRDA
          AEAKSRKGDV PGPCRLFPGG ERGVGSASWR
  301 LSPSEGEPGA GACAETKRFL YCSGGGEQLR
          RSFLLCSLPP SLAGARTLVE TIFLDSKPGP
  361 PGAPRRPRRL PARYWQMRPL FRKLLGNHAR
          SPYGALLRAH CPLPASAPRA GPDHQKCPGV
  421 GGCPSERPAA APEGEANSGR LVQLLRQHSS
          PWQVYGLLRA CLRRLVPAGL WGSRHNERRF
  481 LRNVKKLLSL GKHGRLSQQE LTWKMKVQDC
          AWLRASPGAR CVPAAEHRQR EAVLGRFLHW
  541 LMGAYVVELL RSFFYVTETT FQKNRLFFFR
          KRIWSQLQRL GVRQHLDRVR LRELSEAEVR
  601 QHQEARPALL TSRLRFVPKP GGLRPIVNVG
          CVEGAPAPPR DKKVQHLSSR VKTLFAVLNY
  661 ERARRPGLLG ASVLGMDDIH RAWRAFVLPL
          RARGPAPPLY FVKVDVVGAY DALPQDKLAE
  721 VIANVLQPQE NTYCVRHCAM VRTARGRMRK
          SFKRHVSTFS DFQPYLRQLV EHLQAMGSLR
  781 DAVVIEQSCS LNEPGSSLFN LFLHLVRSHV
          IRIGGRSYIQ CQGIPQGSIL STLLCSFCYG
  841 DMENKLFPGV QQDGVLLRLV DDFLLVTPHL
          TRARDFLRTL VRGVPEYGCQ VNLRKTVVNF
  901 PVEPGALGGA APLQLPAHCL FPWCGLLLDT
          RTLEVHGDHS SYARTSIRAS LTFTQGFKPG
  961 RNMRRKLLAV LQLKCHGLFL DLQVNSLQTV
          FTNVYKIFLL QAYRFHACVL QLPFSQPVRS
1021 SPAFFLQVIA DTASRGYALL KARNAGASLG
          ARGAAGLFPS EAAQWLCLHA FLLKLARHRV
1081 TYSRLLGALR TARARLHRQL PGPTRAALEA
          AADPALTADF KTILD
```

Several conserved regions are identified from the protein sequence annotated in the Protein Data Bank entry for bTERT (Q271D4 TERT_BOVIN) and from molecular modeling algorithm such as Pfam (cdd238826) and included in the NCBI entry.

Several phosphorylation sites are identified in TERT; a serine phosphorylated by PKB/AKT1 (aa 231), a serine phosphorylated by DYRK2 (aa 450) and a tyrosine phosphorylated by SRC-type Tyr-kinase (aa 700). Other important residues, like aa 169 and aa 860, are required for regulating specificity for telomeric DNA and for processivity for primer elongation, and for nucleotide incorporation and primer extension rate respectively, while aa 705 is thought to be implicated in its catalytic activity as it is a magnesium binding site.

One advantage of using lentiviral vectors for gene delivery reside in their ability to deliver long-term stable expression after their integration into the host genome (Gierman, 2007); the infection of both dividing and non-diving cells using the target cell nuclear import machinery (Denning, 2013; Bukrinsky, 1999); and the use of basic molecular biology techniques for its creation, accommodating large transgenes (up to 10 kb) (Matrai, 2010).

Lentiviral vectors are derived from the human HIV-1 virus. Because it is a human pathogen, the latest generation of lentiviruses used for research has several built-in safety considerations. These features comprise the split of the generic material for the necessary components for virus production across 4 different plasmids, (3rd generation of lentivirus):

Lentiviral transfer plasmid encoding the genes of interest: Bos taurus Telomerase Reverse Transcriptase (bTERT, NM_001046242.1) under the CMV promoter and puromycin N-acetyl-transferase gene under the control of the RSV promoter. For safety reasons, transfer plasmids are all replication incompetent and contain an additional deletion in the 3′LTR, rendering the virus “self-inactivating” (SIN) after integration. Moreover, only this lentiviral transfer plasmid contains signals that allow the genetic material encoded in the other plasmids to be packaged into virions, resulting in the absence of genes encoding viral proteins to be packaged.

Packaging plasmids: one plasmid encoding Rev and a second plasmid encoding Gag and Pol proteins; the expression of genes encoding the packaging proteins in separate plasmids relies on a chimeric 5′LTR fused to a heterologous promoter on the transfer plasmid.

Envelop plasmid: the envelope protein, VSV-G, due to its broad tropism is encoded in a separated plasmid. This common envelope protein allows a wide infectivity over a range of species and cell types.

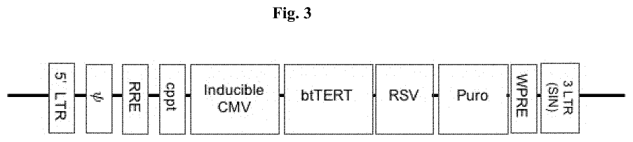

Infectious particles were generated by GenTarget Inc (San Diego, Calif.). The bTERT sequence was subcloned into GenTarget's expression vector (schematically represented in FIG. 3), under the control of an optional inducible CMV promoter that embedded two Tet repressor sites. The vector also contains the puromycin N-acetyl-transferase (Puro) antibiotic marker under RSV promoter. The lentiviral transfer plasmid contains the bTERT sequence under the control of the inducible CMV promoter and the puromycin antibiotic resistant gene under the RSV promoter. Other elements of the plasmid involve the 5′ LTR and 3′ LTR sequences at the end of the viral genome that act as a combined enhancer and promoter, enabling the host cell's RNA polymerase II to start its transcription and to stabilize newly synthesized transcripts by regulating their polyadenylation. The cis-acting viral elements (Ψ, RRE, cppt) encode structural, regulatory, and accessory proteins that are necessary for processing and transport of viral RNAs, as well as the post-transcriptional regulatory elements (WPRE) are included in the transfer plasmid. Trans-acting genes, like rev, gag and pol that are necessary for reverse transcription and integration, and env, for binding to host cells are not included in the lentiviral transfer plasmid for safety measures. The cloned bTERT insert was verified by sequencing analysis. This lentiviral construct constitutively expressed bTERT without the need for induction.

For the generation of the infectious particles, the transfer plasmid was co-transfected with the lentiviral packaging plasmids (catalogue number HT-pack, GenTarget) into 293T cells (catalogue number TLV-C, GenTarget) in DMEM medium according to GenTarget virus production Standard Operating Procedure. Supernatant medium, containing the produced infectious particles, was filtered through 0.45 μm filter, aliquoted and delivered as a frozen product in dry ice. Upon receipt, aliquots were stored at −80° C. until used.

Lentivirus titer were measured using enzyme-linked immunosorbent assay (ELISA) detecting p24 following manufacturer's instructions for the “P24-antigen capture assay kit” (catalogue number 5421, Advanced BioSchece Lab). The lentiviral titer for bTERT CMV-Puro particles was approximately 1×10⁷ IFU/mL, calculated based on the P24 ELISA determination of 1012.18 ng/mL (Report Q#1216, GenTarget).

Control lentiviral particles, encoding the green fluorescent protein (GFP, catalogue number LVP340, GenTarget) in the same backbone of the lentiviral transfer plasmid were used to estimate the transduction efficiency.

Protocols known to the skilled worker for the transduction were followed for the transfer to the genetic material from the lentiviral particles to the progenitor B4M muscle cells, where the initial stages of the transduction mimic the infection with natural viruses and lead to expression of the bTERT and puromycin N-acetyl-transferase, and the insertion of the DNA from the transfer plasmid into the cellular genome. Since none of viral genes are encoded in the lentiviral transfer plasmid, these infections do not generate new viruses; lentiviral particles are normally referred to “replication-deficient” due to this feature.

For the generation of a stable B4M cell line constitutively expressing bTERT, the transfer plasmid contains the selectable marker, puromycin N-acetyl-transferase, that confers antibiotic resistance to the infected host cells. After transduction of muscle progenitor cells, puromycin was added to growth medium, killing off any cells that have not incorporated the lentiviral genome and the cells that did survive can be expanded to create a stable cell line; surviving cells are expected to have integrated the lentiviral genome, contained the genetic information encoded by the genome, and transcribed the proteins (bTERT and puromycin N-acetyl-transferase) in a constitutive manner.

To determine the least amount of the antibiotic puromycin to use for selection of transduced progenitor muscle cells, a kill curve was assay was performed.

Early progenitor muscle cells (B4M cells at passage 2) were seeded in wells of cell culture treated 6-well plates (catalogue number 657 160, Greiner Bio-one) previously coated with gelatin at 500,000 cells per plate in 2 mL of SKGM media. Six hours after seeding, 0.5 mL of antibiotic diluted in media was added, for final concentrations of puromycin of 2, 1, 0.5, 0.25, 0.125 and 0 μg/mL. Plates were incubated undisturbed for 72 h at 37° C., 5% CO₂. Cultures were washed with 2 mL/well DPBS to remove dead cells, growth media was replenished as before, maintaining the corresponding antibiotic concentrations and cultures incubated for further 4 days.

At the end of the selection period, cultures were observed under the microscope. The culture in control well (puromycin concentration of 0 μg/mL) was confluent while no surviving cells were observed in the well with the highest antibiotic concentration. The puromycin concentration needed for selection (0.25 μg/mL) was determined as the lowest concentration of puromycin that kills >90-100% of cells during the selection period (7 days).

Several transduction conditions were tested, including different multiplicity of infection (MOI) values, addition of positively charged molecules that neutralize the charge repulsion between the viral particles, like polybrene (Davis, 2002), DEAE dextran (Denning, 2013) and protamine sulfate (Lin, 2012), in static or centrifugation-enhanced transduction protocols (or spinoculation; Sanyal, 1999), using lentiviral particles expressing the green fluorescent protein (GFP) to assess transduction efficiencies. Higher levels of GFP expression were observed when the spinoculation method was used with a MOI of 20 (data not shown).

Early progenitor B4M muscle cells (at passage 3) were seeded at 250,000 cells per plate in 2 mL of SKGM media. The following day, the culture media was removed and lentiviral dilution (estimating 50,000 cells in each well, at MOI of 20) in 1.5 mL of SKGM media was added for the experimental sample. Control samples were set up in parallel, two samples with GFP-expressing lentiviral particles (at MOI of 20) and three other samples with no lentiviral particles (100 μL of DPBS replacing the volume of infectious particles). The plate was centrifuged at 300 g for 30 min in a swing-bucket rotor (5810R rotor A-4-81 with 4 MTP/Flex bucket, Eppendorf), with the centrifuge previously warmed at 30° C. Six hours after spinoculation, an additional 0.5 mL/well SKGM was added and the plate was incubated undisturbed for 48 h at 37° C., 5% CO₂.

Culture media was removed from transduction plates, and cultures were washed twice with 2 mL/well DPBS to remove any non-incorporated lentiviral particles; spent media and washes were inactivated for the safe disposal of this material. Selection media (SKGM media with 0.25 μg/mL of puromycin) was added to all cultures, except to one of the untransduced cultures that served as a positive control for cell growth which was incubated in SKGM media only.

To estimate the efficiency of the transduction, one of the cultures transduced with the green fluorescent protein (GFP)-expressing lentivirus and one of the un-transduced cultures were dissociated from the plate with 0.3 mL/well of TrypLE Express (catalogue number 12505-010, Thermo Fisher) for 5 min at 37° C., 5% CO₂ after the DPBS wash. Cells were collected with an equal volume of SKGM media.

Percentage of GFP-expressing cells was assessed by flow-cytometry. The untransduced culture was used to set up the gates for single cell suspension and FL-1H positive cells, encompassing <1% of cells from this sample. Approximately 18% of GFP+ cells were observed in the test sample, suggesting a lower transduction efficiency than those observed for other human primary lines (Lin, 2012).

Transduced cultures were kept continuously under selection (SKGM media with 0.25 μg/mL of puromycin) during the expansion stages to enrich the culture with transduced cells that are expressing puromycin N-acetyl-transferase, while the control culture was kept in SKGM media.

Three days after selection, cultures were observed under the microscope and confirmed the growth of the un-transduced cells in normal media to high confluency levels, and the killing of most of the cells of the un-transduced sample with the selection media, suggesting an efficient selection of the transduced cells. For transduced cultures, expressing bTERT or GFP, confluency levels were intermediate to those of the two controls. When cultures reached 70-80% confluency, cells were dissociated and passaged to flasks of increasing dimensions with ratios of approximately 1:3, to expand the selected cells. Progressively, cultures were transferred from 1 well of a 6-well plate (9.6 cm² of surface area) to cell culture treated T-25 flask (25 cm² of surface area) to cell culture treated T-75 flask (75 cm² of surface area) and finally, to cell culture treated T-175 flask (175 cm² of surface area), using 2.5 mL, 5 mL, 10 mL and 25 mL of selection media respectively. The control culture (un-transduced cells growing in SKGM media with no selection) was passaged in a similar manner.

A small fraction of the cell suspension from the cultures after dissociation (approximately 200,000 cells) was used to estimate the percentage of GFP+ cells in the GFP-expressing lentiviral transduced sample. During the first 8 days of selection, the percentage of GFP+ cells increased to 87.2%, with a further increase to 97.5% in the subsequent 11 days in culture. After a total 30 days under selection, the GFP+ cells in the GFP-expressing lentiviral transduced sample reached 99.8%, demonstrating an effective selection of transduced cells. At this point, the culture derived from the transduction with bTERT-expressing lentivirus was considered to be of similar homogeneity and composed mainly of transduced cells. A research cell bank (RCB) was created with the bTERT-transduced cells, denominated B4M-t6 cells.

To test the efficiency of ectopic expression of bTERT in overcoming the replicative senescence seen in cultures of primary progenitor B4M muscle cells, continued culture of bTERT-expressing cells was compared to that of the control, un-transduced cells.

Isolated progenitor muscle cells from the control, un-transduced sample (B4M-t1) or bTERT-transduced cells (B4M-t6) were expanded in gelatin-coated treated cell culture T-75 or T-175 flasks, seeding cells at densities between 2,800 and 3,000 cell/cm², in 10 mL or 25 mL of SKGM media respectively during the first 30 passages. As B4M-t6 cultures proliferated at a faster rate with continued passage, seeding densities were reduced to 2,500 cell/cm² for the next 50 passages and from then onwards, it was further reduced to 1500 cell/cm², while maintaining the original density for the control culture. Cultures were harvested when they reached 70-80% confluency, approximately every 3 to 4 days. Population Doubling Level (PDL) and Population Doubling Time (PDT) were calculated.

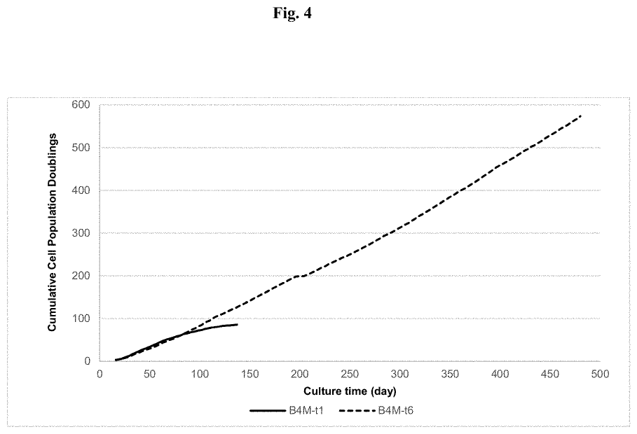

Control and bTERT-expressing progenitor cells proliferated with similar rates (PDT for B4M-t1 of 24.6 h and B4M-t6, 26.4 h) during the first 75 days of the proliferation study, accumulating populating doublings at similar pace as shown in FIG. 4. Approximately after 70 population doublings, control cells showed a sharp decline in the proliferation rate, eventually leading to the proliferation arrest associated to replicate senescence (approximately at PDL of 80). This showed that culture conditions used to expand bovine muscle progenitor cells permitted the extension of their proliferation slightly beyond the well-recognized Hayflick limit (Hayflick 1965), of around 50 to 60 population doublings, at which point replicative senescence was observed.

In stark contrast, a similar decline in the proliferative capacity was not observed for the B4M-t6 line, reaching 80 PDL after 100 days in culture (PDT of 24.5 h); furthermore, with continued passaging, proliferation rate of the B4M-t6 line increased. Seeding densities of this culture were reduced to accommodate for the faster proliferation maintaining a similar passaging regime. After approximately 160 days in culture, B4M-t6 cells have accumulated 150 PDL (average PDL of 22.1 h), almost doubling the proliferative life span of control cells. After 290 days in culture, B4M-t6 cells have doubled 300 times (average PDT of 21.7 h), and they reached 500 PDL after proliferating for 430 days (with PDL of 19.9 h), with continued cell replication and the successful generation of an immortalized bovine line of muscle origin. FIG. 4 shows that B4M-t6 cells have been immortalized as they continue to proliferate for after more than one year of propagation, far surpassing the Hayflick limit.

A selection of markers used for the phenotypic characterization of the isolated progenitor muscle cells as shown in Table 1 was used to confirm the maintenance of the phenotype of these cells during expansion. Culture at passage 16, corresponding to 30 days in culture (PDL of 50) is considered to only contain puromycin-resistant cells, that is, cells that have integrated the lentiviral transfer plasmid and, consequently, express btTERT. B4M-t6 cells at passage 35, reached after approximately 115 days in culture (at 100 PDL) is the stage at which the control cells showed signs of proliferation arrest due to the onset of replicative senescence. B4M-t6 cells at later passages, 72 and 98 (approximately after 250 and 330 days in culture, corresponding to 245 and 365 PDL, respectively) are parallel cultures, maintained under adherent condition, similar to those that were used for the initiation of the suspension adaptation experiments and those that showed anchorage-independent growth.

While some early myogenic markers were detected at the different stages during cell line development, like Myf5 and PAX3, other markers were lost during the prolonged proliferation. Expression of CD82 was detected at the earlier stages of proliferation, while expression of PAX7 was not detected after 250 days in culture. The mature myogenic marker MyHC2 was absent in all the stages, demonstrating that the maintenance of the undifferentiated phenotype for B4M-t6 cells.

Telomere length of B4M-t6 cells were determined at Life Length (Madrid, Spain) using the high throughput (HT) qFISH technique. Briefly, this method uses fluorescence in situ hybridization with a fluorescent Peptide Nucleic Acid probe (PNA) that recognizes three telomere repeats (sequence: Alexa488-OO-CCCTAACCCTAACCCTAA, Panagene); the quantified fluorescent signal captured by a high-content screen system is translated to telomere length by Life Length's algorithm (Canela et al., 2007).

Cryopreserved cells from control B4M and bTERT-transduced B4M-t6 cultures at different stages of expansion were thawed and cell counts (minimum of 1.5×10⁵ cells) and cellular viability (minimum of 60% viability) were determined. Cells were seeded in clear bottom black-walled 384-well plates at the density of 15,000 cells/well with 5 replicates of each sample and 8 replicates of each control cell line. Two identical independent plates were prepared for each set of samples. Cells were fixed with methanol/acetic acid (3/1, vol/vol), treated with pepsin to digest the cytoplasm and the nuclei were processed for in situ hybridization with the PNA probe. After washing, nuclei were stained with DAPI, and the wells were filled up with mounting medium. Plates were stored overnight at 4° C. until imaging.

Quantitative image acquisition and analysis was performed on a High Content Screening Opera System (Perkin Elmer), using the Acapella 1.8 software (Perkin Elmer). Images were captured, using a 40×0.95 NA water immersion objective. UV and 488 nm excitation wavelengths were used to detect the DAPI and Alexa488-conjugated probe signals respectively. With constant exposure settings, 15 independent images were captured at different positions for each well. The nuclei images were used to define the region of interest for each cell, measuring telomere fluorescence intensity of the A488-channel image in all of them. The results of intensity for each foci were exported to the Columbus 2.4.1 software (Perkin Elmer).

The relative fluorescent intensity was calculated by normalizing the fluorescent intensity from the Alexa488-conjugated signal by the nuclear DAPI signal. Telomere length distribution and median telomere length were calculated using Life Length's proprietary algorithm correlating the normalized fluorescent signal from the control cell line to telomere length measurements determined by TRF (Terminal Restriction Fragmentation) in six human lymphocyte cell lines. The TAT assay showed a limit of detection of 800 bp and demonstrated very high specificity of the PNA probe.

The telomere length distribution for control B4M cells isolated from bovine muscle (B4M p0) indicated a median telomere length of approximately 14.6±0.3 kb. Although telomere length varies depending on the animal age and the tissue analyzed, the obtained value correlates well with previous reports for telomere length in several domestic animal species between 10 to 30 kb (Nasir, 2001; Argyle, 2003; Alexander, 2007), and specifically for cows, between 11.0 to 21.0 kb at different ages (Jeon, 2005; Tilesi, 2010). Telomere length of the isolated cells is dependent, not only on the specific tissue and the age of the animal, but is also influenced by the specific breed, as telomere length variations were linked to different breeds (Tilesi, 2010),

To assess the effect of the expression of bTERT, telomere distributions for B4M and B4M-t6 cultures at different stages were assessed and the telomere median length and percentage of very short telomeres (<3 kbp) were calculated. Expanded cells from the isolation (B4M p0), an intermediate passage (B4M p15) and cultures that showed signs of proliferation arrest due to the onset of replicative senescence (B4M p36) from control cultures, together with cultures derived from the B4M-t6 line at similar (B4M-t6 p14 and p36) and later stages (B4M-t6 p60, p86 and p105) were analyzed.

The median telomere length for both control and transduced cultures decreased with time in culture; the reduction in telomere length correlated well with the passage as a function of passage. The attrition rate was higher for the control culture than for cells expressing bTERT.

The effect of proliferation can also be observed in the accumulation of very short telomeres; these are identified as telomeres of length of 3 kb or smaller and represents an approximate telomere length at which the replicative senescence program is triggered. The percentage of very short telomeres determined from the histogram of telomere length distribution was plotted as a function of culture passage and analyzed. The accumulation of shorter telomeres increased more rapidly for the control culture than for B4M-t6; after extensive time in culture (360 days for B4M-t6 p105). Interestingly, the expression of bTERT did not prevent the accumulation of very short telomeres. Despite the presence of short telomeres in B4M-t6 p105 cells, no reduction in the proliferation rate was observed for the B4M-t6 at this stage or at later stages (cultures maintained their proliferation rate for at least 120 days more than those analyzed for telomere length). Without being bound by theory, despite the presence of short telomeres (smaller than 3 kb) which would normally trigger the replicative senescence in bovine cells, another cellular mechanism overrides the former, allowing cells to continue to divide in aeternum regardless of actual telomere length.

Next, transcriptome analysis was performed. 3 μg of RNA, prepared and diluted in RNase-free water to a final volume of 15 μL. RNA quality was assessed on a 2100 Bioanalyzer (Agilent Technologies) using RNA 6000 Nano chips (Agilent Technologies). All samples had an RNA integrity number of between 9.0 and 10.0, and 28S/18S ratio >1. RNA library preparation⁷ and sequencing was performed at BGI (Hong Kong) using the DNBseq sequencing platform.

RNAseq data and bioinformatic work-flow: reads mapped to rRNAs were discarded and reads with low quality, reads with adaptors and reads with unknown bases (N bases more than 5%) were also removed to obtain clean reads (rawdata). Those clean reads were mapped onto the Bos taurus reference genome (GCF_002263795.1_ARS-UCD1.2) using HISAT2 [(Kim, Langmead and Salzberg, 2015); Hierarchical Indexing for Spliced Alignment of Transcripts; HISAT2_2.0.4] to do the mapping step.

Log transformed normalized data by DESeq2 [(Love, Huber and Anders, 2014) DESeq2_1.22.2 with the following parameters >2-fold change and adjusted p-value <0.01] was used for clustering and calculation of Euclidean sample distances, and for identification of DEGs (differentially expressed genes). To discover significant alterations of gene ontology terms and pathways between different sample groups, differentially expressed genes were analyzed using clusterProfiler [(Yu et al., 2012); clusterProfiler_3.10.1] and GAGE [(Luo et al., 2009); gage_2.32.1]. Transcriptome analysis showed that bTERT was expressed in the transduced cells.

### Example 5

### Adaptation of B4M-t6 Cells for Suspension Culture

To minimize the cost and maximize efficiency for large scale production, we have successfully adapted B4M-t6 cell line into suspension culture. A methodology of gradual adaptation was implemented. Cellular adaptation to suspension culture often requires an extended period of time to get adjusted to the new microenvironment and to acquire a healthy appearance and an obvious growth in suspension. B4M-t6 cells at passages between 50-100 were cultured in gelatin-coated (EmbryoMax 1% gelatin in water, Millipore, cat# ES-006-B) T-175 flasks for expansion in Skeletal Muscle Cell Growth Media (abbreviated SKGM, PromoCell, catalog number: C-23060) with 1× Supplement Mix (PromoCell, Catalog number: C-39365). Once sufficient cells were obtained, B4M-t6 cells were transferred to suspension conditions in Thomson Optimum Growth Erlenmeyer flask at a starting cell concentration of between 0.5×10⁶ to 1.0×10⁶ cells/mL. Culture media used for suspension cultures was the same one used for expansion of B4M-t6 cells in attachment, with the addition of two supplements (and internally labelled as SKGMS media): 0.5% (v/v) Pluronic F-68 (Thermo Fisher, catalog number: 24040-032) and 1:100 (v/v) Anti-clumping Agent (abbreviated ACA, Gibco, catalog number 01-0057AE).

B4M-t6 cells were maintained in suspension culture under agitation in 1× supplemented SKGM with 0.5% Pluronic and 1:100 ACA (SKGMS). Antibiotics were not used at any stage of the cell culture. B4M-t6 cells in suspension were cultured in Thomson Optimum Growth Flasks unless otherwise mentioned. The B4M-t6 cells were passaged every 3-4 days and were maintained in a shaking incubator at 37° C., 5% CO₂ in a humidified (70-80%) atmosphere. Briefly, B4M-t6 cells were sub-cultured using spin passage. In the spin passage method, the cells were centrifuged, and the supernatant was discarded. The cell pellet was mechanically dissociated by resuspension with culture media.

For quantification of viable cell density (VCD) and viability (%), 1 mL of B4M-t6 suspension culture was collected. 1 mL of B4M-t6 suspension culture was collected in an Eppendorf tube and micro-centrifuged. The supernatant was discarded or collected for metabolite analysis. The cell pellet was resuspended in 500 μL of TrypLE Express (Gibco) and incubated for 5-8 min at 37° C. on a shaking platform, followed by addition of 500 μL of culture media to neutralize TrypLE Express. The total volume (or the minimum volume of 550 mL per sample) was transferred to sampling cups for the Vi-Cell XR Cell Viability Analyzer (Beckman Coulter). Viable cell density and viability % were quantified using the Vi-Cell analyzer. Nova Flex bioanalyzer (Nova Biomedical, USA) was used to evaluate the pH and concentrations of glucose, glutamine, glutamate, lactate, ammonium, potassium, and sodium. One (1) mL of sample (from previous step) was used for media component and metabolite analyses. The osmolarity of fresh and spent media was measured using OsmoPro osmometer (Advanced Instruments) using 20 μL of sample. Population doubling time (PDT) and Population doubling level (PDL) were calculated.

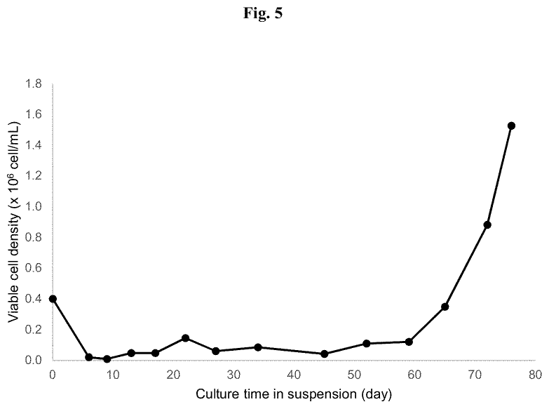

The evolution of B4M-t6 cells towards cultures with proliferation ability in suspension conditions is depicted in FIG. 5. Cells went through a selection process during the first week of culture with a significant dip in cell number, followed by a two-month long stable and constant balance at very low cell concentration. After day 60, proliferation was consistent and regular passaging schedule at each 3-4 days was re-initiated, until reaching viable cell density of 1.53×10⁶ cell/mL. At this point, B4M-t6 cells were adapted to suspension culture and were internally designated as B4M-t6S1 cells. FIG. 5 shows the successful adaptation of B4M-t6 cells from adherent culture to suspension culture. Master working cell banks and master cell banks were prepared by expansion of B4M-t6S1 cells and freezing the cells using commercially available cryopreservation media or SKGMS media to which 10% DMSO has been added.

### EXAMPLE 6

### Adaptation to Serum Free Cultivation

The cells adapted to grow in suspension culture of Example 5 was weaned of animal components, fetuin and FCS/FBS. The transition to serum free cultivation conditions was the use of IMDM media with additional vitamins, lipids, trace elements, and higher concentration of growth factors. The cells were first weaned from fetuin by removing fetuin from the culture medium. Once the cells were adapted to grow without the addition of fetuin, the serum weaning process was started. FBS weaning consisted of multiple steps of serum reduction until growth without the addition of serum was achieved. When good growth is established following a given serum-reduction step was achieved, the serum was reduced further, and the process was repeated.

Once the cells were adapted to grow in IMDM without the addition of fetuin or serum, the cells were cryopreserved by freezing the cells in freezing media, Cryostor CS5 (BioLife Solutions).

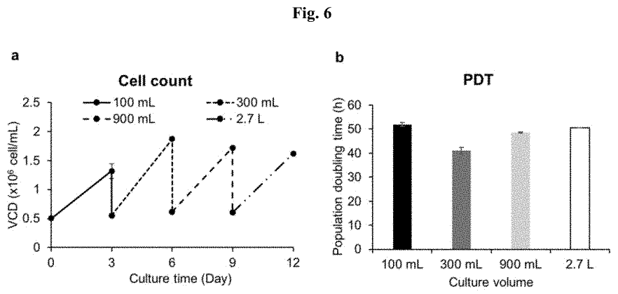

The cells frozen in Cryostor media were next thawed and scaled up in shake flasks. FIG. 6 shows the population doubling times of B. taurus cells adapted to grow in culture media free of animal fetuin and serum. The population doubling time was approximately 50 hours. The cells have been cultivated to achieve over 60 population doublings.

### Example 7

### Wagyu Cells

Myosatellite cells were isolated from muscle tissue obtained from a pure-bred Wagyu cattle from a Japanese breeder as taught in Example 1. Examples of myosatellite cells isolated from Wagyu cattle include B9M and B10M cells. Cell surface marker expression of the Wagyu myosatellite cells were analyzed using the methods taught in Example 2. The expression patterns of CD56 and CD29 were similar to that of the B4M cells. Expression of myogenic markers was characterization by gene expression analysis using the primers listed in Table 1 and results were again similar to the ones found for B4M cells.

The Wagyu myosatellite cells were differentiated into cells with a more mature muscle phenotype as taught in Example 3. A phase contrast microscope image very similar to FIG. 2 (B4M cells) was obtained, showing the formation of thin and elongated multinucleated cells.

The Wagyu myosatellite cells were engineered to express bovine telomerase reverse transcriptase as taught in Example 4, generating an immortalized cell line labelled as B10M-t3.

The B10M-t3 were adapted to grow in suspension culture and in the absence of animal serum in accordance with the methods taught in Example 5.

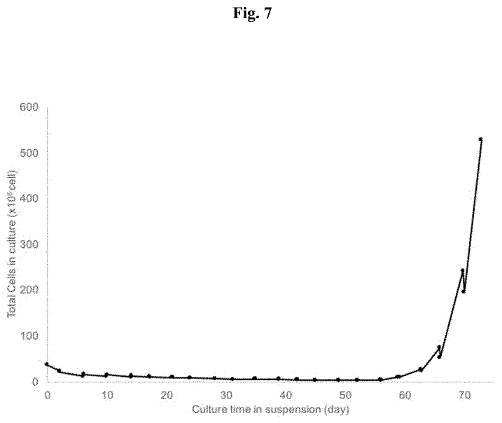

B10M-t3 cells were adapted to serum free cultivation conditions as taught in Example 6. B10M-t3 cells successfully adapted to progressively lower levels of serum in the culture media by supplementation with vitamins, lipids, trace elements and growth factors. By the end of the serum free adaptation process, B10M-t3 cells were growing at similar rates to the cells cultured in serum (approximately 24-30 h of PDT). B10M-t3 cells were then adapted to suspension following the methods in Example 5. The evolution of B10M-t3 cells is shown in FIG. 7. Cells went through a process of no growth until approximately 60 days of culture. After day 60, cell proliferation was evident and consistent and B10M-t3 cells were scaled up to create Research Cell Banks. FIG. 7 shows the successful adaptation of B10M-t3 cells from adherent to suspension cultures.

The Wagyu myosatellite cells were engineered to express bovine telomerase reverse transcriptase as taught in Example 14, generating immortalized cell lines labelled as B9M-SB3 and B9M-SB10.

### EXAMPLE 8

### Cultured Beef Production

Single-use disposable systems are used for the seed expansion and cell growth in the Cultured Beef manufacturing process. The disposal systems with long contact time with the culture media include shake flasks, Wave Bags, media hold bags and eventually stirred-tank reactor bags for the large scale 500 L bioreactor. The extractables and leachable profiles of these systems are extensively validated by the vendors and the detailed guides provided by these vendors are reviewed at Eat JUST and available upon request,

B4M-t6S1 cells, immortalized myoblast cells adapted for growth in suspension culture, were thawed from the MCB, placed in culture in disposable sterile shaking flasks and scaled-up to 25 L Wavebag bioreactor cultures. Briefly, every 3-4 days, cells were sampled, the whole culture volume was centrifuged and resuspended in fresh SKGMS media, being expanded into larger-volume flasks with adjusted volume to bring viable cell density back to 0.3-0.4×10⁶ cell/mL. Cell culture conditions are listed in sections below.

A single use 50 L wave bioreactor was inoculated with cell culture from four 5 L shake flasks and the cell culture level supplemented to with fresh SKGMS media in a 1:3 split ratio. On the day of inoculation, seeding density was at 0.23×10⁶ cell/mL. The culture temperature and pH were controlled at 37° C. and 7.4 ±0.3 respectively. pH was maintained within physiological range (7.4±0.3) using 5 N NaOH and carbon dioxide (CO₂). The DO concentration was controlled at a set point of 40% of air saturation. After a 4-day batch cell culture, the entire contents of the Wavebag were distributed into different 1 L pre-sterilized centrifuge bottles for downstream cell harvest.

To harvest cell paste, the cell suspension (slurry) was aseptically drained from the WaveBag into pre-sterilized 1 L centrifuge bottles. The slurry was centrifuged in a centrifuge at 3000×g for 15 min. 25-50 mL aliquots of cell culture supernatant were collected for future cell paste release testing.

The beef cell pellet (slurry) from the centrifugation process was washed twice, each time by resuspending the pellet with a five volume of 0.45% NaCl (w/v) solution. The final wash solution was tested for insulin and Pluronic F-68 to confirm the effectiveness of washing. Pluronic F-68 in the wash solutions was measured using colorimetric cobalt thiocyanate method. Pluronic F-68 in the sample forms a complex with cobalt thiocyanate that sediments upon centrifugation. The precipitate was dissolved in acetone and the color intensity was correlated to the Pluronic F-68 concentration in the linear range for quantification. Insulin in the wash solutions is detected and quantified using Insulin Quantikine ELISA kit (R&D Systems, Cat# DINS00) with high sensitivity and specificity for human, canine and porcine insulin. After the final wash, the beef cell paste contained <8 pmol/L insulin and less than 0.01% Pluronic F-68.

### EXAMPLE 9

### Culture in 500 L Bioreactor

The contents of a Wave Bag (25 L, 50 L, or 100 L) are aseptically transferred to a large-scale bioreactor (total volume of 700 L with maximum working volume of 500 L) with 100 L of initial culture media (with a 1:3 to 1:6 split ratio to a total volume of 125 L).

After 3 days (+/−0.5 days) of culture, the media volume is increased to 500 L by the addition of 375 L new culture media and continued for an additional 3 days (+/−0.5 days). Cultures are sampled regularly to determine cell number and viability. Bioreactor culture is monitored off-line for pH, lactate, glucose, glutamine and glutamate levels.

### EXAMPLE 10

### Testing Safety of Cells for Bacteria And Viruses

Safety and efficacy of the cells is checked at all stages of growth and harvesting of the cells. Cultured Bos taurus cells are evaluated for presence of viral, yeast, and bacterial adventitious agents.

The cells are analyzed for the presence of bacteria using protocols from the FDA's Bacteriological Analytical Manual (BAM).

Total Plate Count (TPC) is synonymous with Aerobic Plate Count (APC). As indicated in the US FDA's Bacteriological Analytical Manual (BAM), Chapter 3, the assay is intended to indicate the level of microorganism in a product. Briefly, the method involves appropriate decimal dilutions of the sample and plating onto non-selective media in agar plates. After incubating for approximately 48 hours, the colony forming units (CFUs) are counted and reported as total plate count.

Yeast and mold are analyzed according to methodology outlined in the US FDA Bacteriological Analytical Manual (BAM), Chapter 18. Briefly, the method involves serial dilutions of the sample in 0.1% peptone water and dispensing onto a petri plate that contains nutrients with antibiotics that inhibit microbial growth but facilitate yeast and mold enumeration. Plates are incubated at 25° C. and counted after 5 days. Alternately, yeast and mold are analyzed by using ten-fold serial dilutions of the sample in 0.1% peptone water and dispensing 1 mL onto Petrifilm that contains nutrients with antibiotics that facilitate yeast and mold enumeration. The Petrifilm is incubated for 48 hours incubated at 25 or 28° C. and the results are reported as CFUs.

Escherichia coli and coliform are analyzed according to methodology outlined in the US FDA Bacteriological Analytical Manual (BAM), Chapter 4. The method involves serial decimal dilutions in lauryl sulfate tryptone broth and incubated at 35° C. and checked for gas formation. Next step involves the transfer from gassing tubes (using a 3 mm loop) into BGLB broth and incubated at 35° C. for 48+/−2 hours. The results are reported as MPN (most probable number) coliform bacteria/g.

Streptococcus is analyzed using CMMEF method as described in chapter 9 of BAM. The assay principle is based on the detection of acid formation by Streptococcus and indicated by a color change from purple to yellow. KF Streptococcus agar medium is used with triphenyl tetrazolium chloride (TTC) for selective isolation and enumeration. The culture response is reported as CFUs after incubating aerobically at 35+/−2° C. for 46-48 hours.

Salmonella is analyzed according to methodology outlined in the US FDA Bacteriological Analytical Manual (BAM), Chapter 5. Briefly, the analyte is prepared for isolation of Salmonella then isolated by transferring to selective enrichment media, the plated onto bismuth sulfite (BS) agar, xylose lysine deoxycholate (XLD) agar, and Hektoen enteric (HE) agar. This step is repeated with transfer onto RV medium. Plates are incubated at 35° C. for 24+/−2 hours and examined for presence of colonies that may be Salmonella. Presumptive Salmonella are further tested through various methodology to observe biochemical and serological reactions of Salmonella according to the test/substrate used and result yielded.

Cultured Bos taurus cells are considered acceptable for Mycoplasma for example, if a minimum 3% of randomly selected and tested cell vials from each bank are thawed and their culture supernatants provide a negative result using the MycoAlert™ Mycoplasma Detection Kit. Following the kit guidelines, the tested samples are classified according to the ratio between Luminescence Reading B and Luminescence Reading A: Ratio <0.9 Negative for Mycoplasma; 0.9<Ratio<1.2 Borderline (required retesting of cells after 24 hours); Ratio>1.2 Mycoplasma contamination.

Viral assessment can be performed by analyzing adventitious human virus and bacterial agents through an Infectious Disease Polymerase Chain Reaction (PCR) performed in-house or by a third-party (Charles River Research Animal Diagnostic Services)—Human Essential CLEAR Panel; Bacteria Panel.

Bos taurus cell banks are considered valid for viral assessment if a minimum of 3% of independent cell vials from the tested bank are thawed and their cell pellets provide a negative result for the full panel of adventitious agents.

Cultured Bos taurus cells are considered approved for absence of adventitious human viral and bacterial agents if the independent cell pellets from each cell bank are negative for the entire human panels.

Detection of adventitious contaminations was performed by testing B4M-t6S1 cells. B4M-t6S1 cells are considered valid for viral assessment if a minimum of 0.4×\sqrt{n} of randomly selected and tested cryovials from each bank of cells (of “n” bank size) are thawed and their cell pellets provide a negative result for the full panel of adventitious agents listed in Table 2. In Table 2, Negative (absence of virus/bacteria) is noted with Positive (presence of virus/bacteria) is noted with. As can be seen in Table 2, no adventitious agents were present in the B4M-t6S1 cells.

TABLE 2
Panel of human adventitious agents tested in B4M-t6S1 cells.
HUMAN ESSENTIAL CLEAR PANEL

| | MCB: | MWCB: |
|---|---|---|
| | BR02-101519B | BR02-012820 |
| Adeno-associated virus | — | — |
| BK virus | — | — |
| Epstein-Barr virus | — | — |
| Hepatitis A virus | — | — |
| Hepatitis B virus | — | — |
| Hepatitis C virus | — | — |
| Herpes Simplex 1 PCR | — | — |
| Herpes Simplex 2 PCR | — | — |
| Herpesvirus type 6 | — | — |
| Herpesvirus type 7 | — | — |
| Herpesvirus type 8 | — | — |
| HIV-1 | — | — |
| HIV-2 | — | — |
| HPV-16 | — | — |
| HPV-18 | — | — |
| Human cytomegalovirus | — | — |
| Human Foamy virus | — | — |
| Human T-lymphotropic virus | — | — |
| John Cunningham virus | — | — |
| Parvovirus B19 | — | — |
| Mycoplasma Genus PCR | — | — |
| Mycoplasma pulmonis PCR | — | — |

### Example 11

### Bovine Food Product Composition

A representative food product composition is described below (by weight percentage) in Table 3.

TABLE 3
Example bovine food product composition.

| Ingredient | % by weight |
|---|---|
| Water | 20-40 |
| Bos taurus Cell paste | 25-50 |
| Mung bean | 10-20 |
| Fat | 5-20 |
| transglutaminase | 0.0001-0.0125 |

### Example 12

### Identity And Purity Of Bovine Cells Cultivated In Vitro

The B4M-t6S1 cells used for cultured beef production were analyzed by PCR internally to confirm their identity and purity. This was confirmed by an external genotype sequencing analysis of the amplified products from the PCR reaction at Quintara Biosciences (CA 94545, USA)

Briefly, PCR amplification was performed using primers designed to amplify highly conserved regions of the Cytochrome C Oxidase Subunit 1 gene (COX1, NC_006853.1). These primers contain degenerate bases that allow the amplification of vertebrate (non-fish) sequences; the COX1 locus retains enough sequence conservation through evolutionary history that allows the identification of organisms, and at the same time, has enough sequence diversity that permits to differentiate organisms to at least the family level. The “bar-coding” strategy allows the use of single pair of primers to verify the species identity of all mammalian (and avian) species.

Quality of amplified 700-bp fragment DNA was checked in an agarose gel and then sent for sequencing to an external service. Sequencing results were compared to published mammalian sequences in databases to confirm species identity.

To establish bovine species identity, PCR analysis was performed on isolated genomic DNA from B4M-t6S1 MCB or MWCB samples and a sample from cells with known bovine species identity (positive control, e.g., genomic DNA isolated from an early passage of the same line previously verified by sequencing).

Primer sequences used for amplification and sequencing were as follows. VF1d_t1: TGTAAAACGACGGCCAGTTCTCAACCAACCACAARGAYATYGG (SEQ ID NO: 42) VR1d_t1: CAGGAAACAGCTATGACTAGACTTCTGGGTGGCCRAARAAYCA (SEQ ID NO: 43)

Sequenced amplicons were then compared with sequences deposited in public databases (NCBI), particularly the one for Bos taurus isolate CDY472 mitochondrion, complete genome MN200938.1. B4M-t6S1 cells are considered validated as bovine cells when the percentage of alignment with the published bovine sequence is higher than 95% for a minimum of 0.4×\sqrt{n} samples of randomly selected and tested cryovials from each bank (of “n” bank size).

B4M-t6S1 cell pellets from independent vials of the MCB:BRO2-101519B and MWCB:BRO2-012820 were collected aseptically. DNA was extracted, and the PCR reaction was performed using the PCR primers indicated in Table 1. Afterwards, the amplicons were run on agarose gels to assure amplicon purity and size. The DNA fragment was amplified by PCR reaction with the expected size. After confirming the successful reaction, amplicons were purified, and the samples were shipped for DNA Sanger sequencing at Quintara Biosciences. Sequence alignment between the genotyped amplicon and the published bovine consensus sequence (from National Center of Biotechnology Information) was performed using the online software tool “Align Sequences Nucleotide BLAST” available at blast.ncbi.nlm.nih.gov. The fragment was of the expected size and sequences revealed 100% sequence alignment to the published beef sequence. The high level of homology between the amplified product and the public genomic databases of Bos taurus confirmed the identity of B4M-t6S1 cells banked in MCB and MWCB as bovine cells.

### Example 13

### Reducing Lactate Production

During cell growth, metabolite (e.g., lactate, ammonia, amino acid intermediates) accumulation have been shown to be detrimental to cell growth and productivity at certain concentrations (Claudia Altamirano et al., 2006; Freund & Croughan, 2018; Lao & Toth, 1997; Pereira et al., 2018). In a fed-batch process the accumulation of lactate causes a decrease in culture pH requiring the addition of alkali to maintain pH at setpoint or physiological range. Negatively, the addition of alkali causes an increase in the osmolality of the media and it has been shown that higher osmolality levels strongly inhibit the growth and protein production of most cell lines (Christoph Kuper et al., 2007; Kiehl et al., 2011; McNeil et al., 1999).

The major route of lactate accumulation is the interconversion of pyruvate to lactate which is catalyzed by lactate dehydrogenase (LDH). In mammalian cells, studies have shown that LDH exist either as homo- or hetero-tetramers with a subunit A or B, encoded by LDHA or LDHB respectively (Urbańska & Orzechowski, 2019). Moreover, it has been shown that LDHA catalyzes the forward reaction (pyruvate to lactate) and LDHB catalyzes the backward reaction (lactate to pyruvate). LDHA play a key role in the Warburg effect that occurs in cell lines that do not drive the breakdown of pyruvate through the citric acid cycle, producing lactate from pyruvate even in the presence of oxygen

Oxamate, an analogue of pyruvate, is a strong competitive LDHA inhibitor halting the Warburg effect by channeling much of the breakdown of glucose through the tricarboxylic acid (TCA) cycle—a much more energy efficient process (Wang et al., 2019). However, the use of this molecule inhibits cell proliferation which is a key factor at the earlier stage of production for most industrial mammalian cell lines (Kim et al., 2019; Wang et al., 2019).

Bos taurus cells are cultivated in suspension culture supplemented with a desired amount of bovine serum or no serum. Different concentrations of sodium oxamate are tested: 1, 3, 5, 10, 30, 60, 100, and 200 mM, and production of lactate, glucose consumption, cell growth rates and cell density are measured.

The specific rates were calculated using daily viable cell concentration and metabolite concentrations for the duration of the cell culture. Specific net growth rates (μ_{N}) were calculated as a change in VCD over a time interval t₁ to t₂ using equation (1):

$${μ}_{N} = \frac{In [ \frac{VCD 2}{VCD 1} ]}{t 2 - t 1} \qquad ( Equation 1 )$$

Specific glucose consumption rate (qGluc) or specific lactate production rate (qLac) were determined using equation (2), where P is glucose or lactate concentration:

$$qGluc or qLac = {μ}_{N} ( \frac{P 2 - P 1}{VCD 2 - VCD 1} ) \qquad ( Equation 2 )$$

Viable cell density (VCD) and viability are determined by the trypan blue exclusion method using the Vi-cell™ (Beckman Coulter) from 1 mL daily samples taken from shake flask cell culture. Gas and pH values including metabolite (glucose, lactate glutamine, glutamate, ammonium) concentrations are measured using the Bioprofile Flex analyzer (Nova Biomedical). Osmolality is measured using the OsmoPro Multi-Sample Micro-Osmometer (Advanced Instruments) which employs the freezing point technology.

Bos taurus cells treated with different concentrations of sodium oxamate (1, 3, 5 and 10 mM), including untreated control cells, are cultured in a batch mode using duplicate shake flasks for 3 days.

### Example 14

### Alternative Sugars

The impact of alternative sugars (mannose, fructose and galactose) on the growth and metabolism of an in-house Bos taurus cells grown in suspension cultures containing a desired amount of FBS or no serum is determined. Specific net growth rate (μ_{N}) and Specific glucose consumption rate (qGluc) or specific lactate production rate (qLac) are calculated according to equation 1 or 2 as disclosed in Example 13.

Suspension cultures as described herein are cultivated using 3 g/L of the respective sugars were added from day 0 and cultured in a batch mode up to day 3. On day 3 after sampling, an additional 3 g/L of each sugar is added to the respective flasks.

The effect of combining glucose, mannose and fructose on growth and lactate production is also determined. Using a design-of-experiment (DOE) approach, 17 batch shake flask runs are carried out evaluating various combinations of concentrations of glucose, mannose and fructose as energy sources for suspension Bos taurus cells. The experimental design used includes 3 factors (glucose, mannose and fructose) and 4 levels (0, 0.5, 1.5 and 3.0 g/L). At days 1, 2 and 3, the VCD under each condition is determined to identify the best combination of sugars to maximize cell density.

### Example 14

### Transfection of Myosatellite Cells with Sleeping Beauty (SB) and PiggyBac Vectors

In addition to lentiviral transduction as disclosed in Example 4, an alternative non-viral method to induce long-term stable bTERT expression is utilizing transposons. Transposons (also refered to as “jumping genes”, or “transposable elements”) are DNA sequences that can move from one location to another within the genome (Pray, L. (2008) Transposons: The jumping genes. Nature Education 1(1):204). Transposons are found in almost all organisms (SanMiguel, P., et al. Nested retrotransposons in the intergenic regions of the maize genome. Science 274, 765-768 (1996)). Transposons have been developed as a genetic engineering tool for stable gene transfer. The mechanism of transposition is summarized in “cut-and-paste” steps. First, the transposase recognizes and binds to specific sequences, called directed repeats (DRs) located in the inverted terminal repeats (ITRs) of the transposon. Once bound, the transposase “cuts” the transposon sequence from the genomic DNA (gDNA) of the host and form a complex with the removed DNA fragment. This complex moves to a new location, opens the gDNA backbone to insert (“paste”) the fragment of the transposon in to gDNA. Such insertion is mediated by the non-homologous end joining (NHEJ) mechanism by the double strand break repair system.

Sleeping Beauty (SB) and PiggyBac (PB) are vectors are available commercially and can be used as transposons in genetic engineering. The vectors are designed to include the DRs/ITRs regions with the gene of interest (GOI) located in-between DRs and ITRs. SB and PB vectors are often transfected into the cells along with the transposase enzymes. Once inside the host cell, the transposase will remove the GOI from the vectors and move it into the host's gDNA. The GOI is integrated into the host's genome. The SB system is believed to be a safer alternative to viral vectors due to its non-pathogenic origin (e.g., lentiviral based vectors), and exhibits higher transposition activity, lower enhancer activity, and minimal epigenetic induction at the insertion site. PB transduction systems are generally more efficient than SB system with larger cargo (>7 kb) capacity and also can include an excision site that allows removal of the PB vector sequences from the host cell post-transfection.

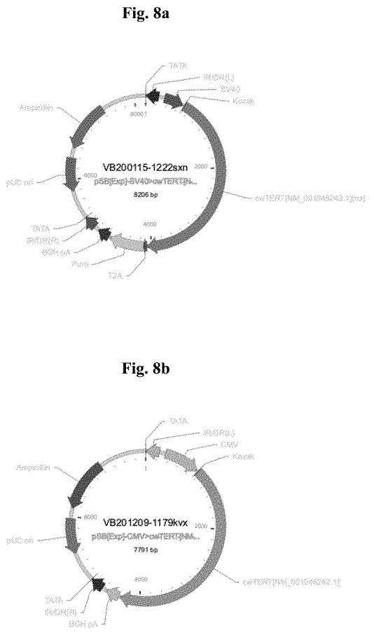

SB and PB vectors for expressing bTERT of SEQ ID NO: 41was were designed by synthesized at VectorBuilder (VB) Inc. The representative schematic of the SB vectors are presented in FIGS. 8a and 8b, respectively. A GOI for expressing the bTERT sequence of SEQ ID NO: 41was subcloned into VB's vectors with the puromycin N-acetyl-transferase (pac) marker under an SV40 promoter or without of the pac marker under a CMV promoter.

The SB vector for containing the bTERT polynucleotide was transfected into B9M Wagyu myoblast cells for insertion into the genomic DNA of the B9M Cells.

For the generation of a stable Wagyu B9M cell line constitutively expressing bTERT (B9M-tert-puro) via antibiotic selection, the vector contains the selectable marker, puromycin N-acetyl-transferase, that confers antibiotic resistance to the transfected host cells. After transfection of muscle progenitor cells, puromycin was added to growth medium, selecting for cells that have incorporated the GOI from the SB vectors. Cells that survived puromycin selection were selected and expanded to create a stable cell line; surviving cells integrated the GOI into the genome, and expressed the proteins btTERT and puromycin N-acetyl-transferase in a constitutive manner.

For the generation of a stable B9M cell line constitutively expressing bTERT without also expressing an antibiotic selection (B9M-tert), the vector did not contain an antibiotic selectable marker that confers antibiotic resistance to the transfected host cells. After transfection of B9M cells, single cell cloning was performed where single individual cells were selected and seeded into each well of a 96-well plate manually based on cell counts or by single cell cloning equipment such as CSight (Molecular Devices, LLC). Colonies that arose from single cells were expanded. RNAs from these colony-derived populations were collected and screened for bTERT expression. Clones with significantly higher (>100-fold) bTERT expression were selected for cryopreservation. The clone with the highest expression of bTERT was selected for expansion and were passaged for long-term proliferation to validate the immortal status of the clonal cells and continued expression of bTERT. Clonal cells integrated the GOI into the genome, and expressed bTERT in a constitutive manner.

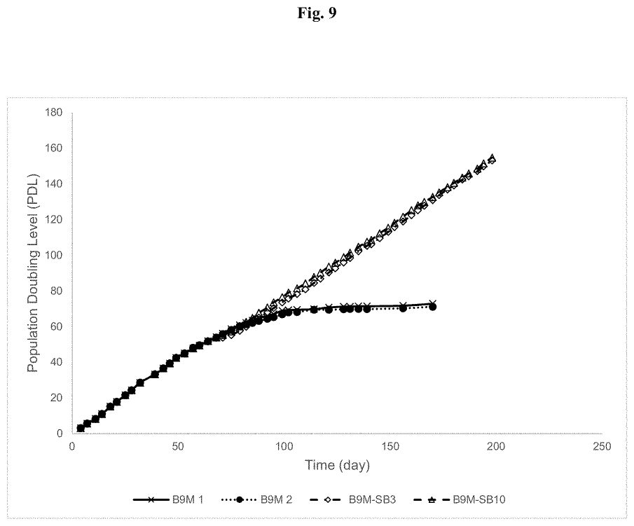

FIG. 9 shows that the population doubling of control B9M cells that were not transfected with SB reached about 60 doublings after 100 days. The data in FIG. 9 is from cells cultivated under adherent culture conditions as described in Example 4. In contrast, B9M-tert, identified as B9M-SB3 and B9M-SB10 cells, that express bTERT were immortalized and reached 160 doublings after 170 days and continued to grow after 170 days in culture.

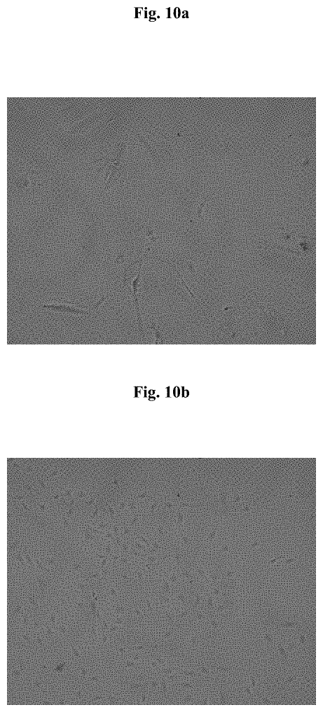

FIG. 10a is a photomicrograph of control B9M cells that are not transfected with SB vectors. FIG. 10b is a photomicrograph of B9M-tert cells that are immortalized by transfection with SB vectors that encode bTERT. FIG. 10a shows that the morphology of the non-transfected cells is enlarged and flat, with signs of cell death. FIG. 10b shows that the immortalized cells are smaller and form more compact colonies that sustain long-term proliferation and display fibroblast-like morphology.

### REFERENCES

Lawler A & Adler J. 2012. Smithsonian Magazine. June. Available at http://www.smithsonianmag.com/history/how-the-chicken-conquered-the-world-87583657/.

USDA Fact Sheets—Poultry Preparation. Focus on: Chicken. Available at http://www.fsis.usda.gov/Fact_Sheets/Chicken_Food_Safety_Focus/index.asp.

Gorman J. 2016. Chickens Weren't Always Dinner for Humans. NY Times. Jan. 18, 2016. Available at www.nytimes.com/2016/01/19/science/chickens-werent-always-dinner-for-humans.html.

English D R, MacInnis R J, Hodge A M, Hopper J L, Haydon A M, Giles G G. 2004. Red meat, chicken, and fish consumption and risk of colorectal cancer. Cancer Epidemiology and Prevention Biomarkers. 13(9):1509-14.

Sinha R, Cross A J, Graubard B I, Leitzmann M F, Schatzkin A. 2009. Meat intake and mortality: a prospective study of over half a million people. Archives of internal medicine. 169(6):562-71; Hu F B, Rimm E B, Stampfer M J, Ascherio A, Spiegelman D, Willett W C. 2000. Prospective study of major dietary patterns and risk of coronary heart disease in men-. The American journal of clinical nutrition. 72(4):912-21.

International Agency for Research on Cancer (IARC). 2018. Monographs on the Evaluation of Carcinogenic Risks to Humans. Volume 114. Red Meat and Processed Meat. IARC, Lyon, France.

Physicians Committee for Responsible Medicine (PCRM). 2013. Letter to The Honorable Sanford Bishop, US Congress, dated Mar. 14, 2013.

Altamirano, Claudia, Illanes, A., Becerra, S., Cairó, J. J., & Gòdia, F. (2006). Considerations on the lactate consumption by CHO cells in the presence of galactose. Journal of Biotechnology, 125(4), 547-556. https://doi.org/10.1016/j.jbiotec.2006.03.023

Freund, N. W., & Croughan, M. S. (2018). A simple method to reduce both lactic acid and ammonium production in industrial animal cell culture. In International Journal of Molecular Sciences (Vol. 19, Issue 2). https://doi.org/10.3390/ijms19020385

Lao, M. S., & Toth, D. (1997). Effects of ammonium and lactate on growth and metabolism of a recombinant Chinese hamster ovary cell culture. In Biotechnology Progress (Vol. 13, Issue 5, pp. 688-691). https://doi.org/10.1021/bp9602360

Pereira, S., Kildegaard, H. F., & Andersen, M. R. (2018). Impact of CHO Metabolism on Cell Growth and Protein Production: An Overview of Toxic and Inhibiting Metabolites and Nutrients. Biotechnology Journal, 13(3), 1-13. https://doi.org/10.1002/biot.201700499

Christoph Kuper, Franz-X. Beck, & Wolfgang Neuhofer. (2007). Osmoadaptation of Mammalian Cells—An Orchestrated Network of Protective Genes. Current Genomics, 8(4), 209-218. https://doi.org/10.2174/138920207781386979

Kiehl, T. R., Shen, D., Khattak, S. F., Jian Li, Z., & Sharfstein, S. T. (2011). Observations of cell size dynamics under osmotic stress. Cytometry Part A, 79 A(7), 560-569. https://doi.org/10.1002/cyto.a.21076

McNeil, S. D., Nuccio, M. L., & Hanson, A. D. (1999). Betaines and related osmoprotectants. Targets for metabolic engineering of stress resistance. Plant Physiology, 120(4), 945-949. https://doi.org/10.1104/pp.120.4.945

Urbańska, K., & Orzechowski, A. (2019). Unappreciated role of LDHA and LDHB to control apoptosis and autophagy in tumor cells. International Journal of Molecular Sciences, 20(9), 1-15. https://doi.org/10.3390/ijms20092085

Wang, Z., Nielsen, P. M., Laustsen, C., & Bertelsen, L. B. (2019). Metabolic consequences of lactate dehydrogenase inhibition by oxamate in hyperglycemic proximal tubular cells. Experimental Cell Research, 378(1), 51-56. https://doi.org/10.1016/j.yexcr.2019.03.001

Kim, E. Y., Chung, T. W., Han, C. W., Park, S. Y., Park, K. H., Jang, S. B., & Ha, K. T. (2019). A Novel Lactate Dehydrogenase Inhibitor, 1-(Phenylseleno)-4-(Trifluoromethyl) Benzene, Suppresses Tumor Growth through Apoptotic Cell Death. Scientific Reports, 9(1), 1-12. https://doi.org/10.1038/s41598-019-40617-3

Lu, Q. Y., Zhang, L., Yee, J. K., Go, V. L. W., & Lee, W. N. (2014). Metabolic consequences of LDHA inhibition by epigallocatechin gallate and oxamate in MIA PaCa-2 pancreatic cancer cells. Metabolomics, 11(1), 71-80. doi.org/10.1007/s11306-014-0672-8

Alexander, M. S. et al. (2016) “CD82 Is a Marker for Prospective Isolation of Human Muscle Satellite Cells and Is Linked to Muscular Dystrophies,” Cell Stem Cell. Cell Press, 19(6), pp. 800-807. doi: 10.1016/j.stem.2016.08.006.

Alonso-Martin, S. et al. (2016) “Gene expression profiling of muscle stem cells identifies novel regulators of postnatal myogenesis,” Frontiers in Cell and Developmental Biology. Frontiers Media S.A., 4(JUN). doi: 10.3389/fcell.2016.00058.

Canela, A. et al. (2007) “High-throughput telomere length quantification by FISH and its application to human population studies,” Proceedings of the National Academy of Sciences of the United States of America. National Academy of Sciences, 104(13), pp. 5300-5305. doi: 10.1073/pnas.0609367104.

Glaser, J. and Suzuki, M. (2018) “Skeletal Muscle Fiber Types in Neuromuscular Diseases,” in Muscle Cell and Tissue-Current Status of Research Field. InTech. doi: 10.5772/intechopen.79474.

Halfon, S. et al. (2011) “Markers distinguishing mesenchymal stem cells from fibroblasts are downregulated with passaging,” Stem Cells and Development, 20(1), pp. 53-66. doi: 10.1089/scd.2010.0040.

Kim, D., Langmead, B. and Salzberg, S. L. (2015) “HISAT: A fast spliced aligner with low memory requirements,” Nature Methods. Nature Publishing Group, 12(4), pp. 357-360. doi: 10.1038/nmeth.3317.

Love, M. I., Huber, W. and Anders, S. (2014) “Moderated estimation of fold change and dispersion for RNA-seq data with DESeq2,” Genome Biology. BioMed Central Ltd., 15(12), p. 550. doi: 10.1186/s13059-014-0550-8.

Luo, W. et al. (2009) “GAGE: Generally applicable gene set enrichment for pathway analysis,” BMC Bioinformatics. BioMed Central, 10(1), p. 161. doi: 10.1186/1471-2105-10-161.

Sastry, S. K. and Horwitz, A. F. (1993) “Integrin cytoplasmic domains: mediators of cytoskeletal linkages and extra- and intracellular initiated transmembrane signaling,” Current Opinion in Cell Biology. Curr Opin Cell Biol, 5(5), pp. 819-831. doi: 10.1016/0955-0674(93)90031-K.

Uezumi, A. et al. (2014) “Identification and characterization of PDGFR+mesenchymal progenitors in human skeletal muscle,” Cell Death and Disease. Nature Publishing Group, 5(4), pp. e1186-e1186. doi: 10.1038/cddis.2014.161.

Verdijk, L. B. et al. (2014) “Satellite cells in human skeletal muscle; From birth to old age,” Age. Springer Science and Business Media Netherlands, 36(2), pp. 545-557. doi: 10.1007/s11357-013-9583-2.

Yablonka-Reuveni, Z. and Nameroff, M. (1987) “Skeletal muscle cell populations—Separation and partial characterization of fibroblast-like cells from embryonic tissue using density centrifugation,” Histochemistry. Springer-Verlag, 87(1), pp. 27-38. doi: 10.1007/BF00518721.

Yang, Y. et al. (2014) “CD29 of human umbilical cord mesenchymal stem cells is required for expansion of CD34+ cells,” Cell Proliferation. Blackwell Publishing Ltd, 47(6), pp. 596-603. doi: 10.1111/cpr.12130.

Yu, G. et al. (2012) “ClusterProfiler: An R package for comparing biological themes among gene clusters,” OMICS A Journal of Integrative Biology, 16(5), pp. 284-287. doi: 10.1089/omi.2011.0118.

1. A cell of the genus Bos, wherein the cell is adapted to grow in growth medium that comprises low-serum or no serum.

2. The cell of the claim 1, wherein the cell is an immortalized cell (non-tumorigenic cell).

3. The cell of claim 1, wherein the serum is calf serum or fetal bovine serum.

4. (canceled)

5. (canceled)

6. The cell of claim 1, wherein the cell is cultivated in a growth medium that comprises less than 1% serum.

7. The cell of claim 1, wherein the cell is cultivated in a growth medium that does not comprise serum.

8. The cell of claim 1, wherein the cell is a muscle cell or a fat cell.

9. The muscle cell of claim 8, wherein the muscle cell endogenously expresses a cell surface receptor selected from the group consisting of CD29, CD56, and CD82.

10. The muscle cell of claim 8, wherein the muscle cell endogenously expresses a transcription factor selected from the group consisting of Pax3, Pax7, Myf5, Mrf4, MyoD, and MyoG.

11. The muscle cell of claim 8, wherein the muscle cell does not endogenously express desmin or myosin heavy chain 2 (MyHC2).

12. (canceled)

13. (canceled)

14. A method of cultivating cell of the genus Bos, the method comprising:
adapting the cell to grow in growth medium that comprises low-serum or no serum.

15. The method of claim 14, wherein the cell is an immortalized cell (non-tumorigenic cell).

16. The method of claim 14, wherein the serum is calf serum or fetal bovine serum.

17. (canceled)

18. The method of claim 14, wherein the growth medium comprises less than 1% serum.

19. The method of claim 14, wherein the growth medium comprises no serum.

20. The method of claim 14, wherein the cell is a muscle cell or a fat cell.

21. The method of claim 20, wherein the muscle cell endogenously expresses a cell surface receptor selected from the group consisting of CD29, CD56, and CD82.

22. The method of claim 20, wherein the muscle cell endogenously expresses a transcription factor selected from the group consisting of Pax3, Pax7, Myf5, Mrf4, MyoD, and MyoG.

23. The method of claim 20, wherein the muscle cell does not endogenously express desmin or myosin heavy chain 2 (MyHC2).

24. (canceled)

25. (canceled)

26. The method of claim 14 , wherein the growth medium comprises one or more of growth factors, fatty acids, proteins, elements, and small molecules.

27. (canceled)

28. (canceled)

29. The method of claim 26, wherein the protein comprises transferrin.

30. The method of claim 26, wherein the element comprises selenium.

31. The method of claim 26, wherein the small molecule is ethanolamine.

32. (canceled)

33. The method of claim 14, wherein the cells are cultivated as a suspension culture.

34. (canceled)

35. A method of producing food product, the food product comprising cells of the genus Bos, the method comprising the steps of:
a. culturing the cells in vitro in a growth medium that comprises low or no serum;
b. recovering the cells from the growth medium; and
c. formulating the recovered cells into an edible food product.

36. (canceled)

37. The method of claim 35, wherein the cells are cultivated in a growth medium that does not comprise serum.

38. (canceled)

39. (canceled)

40. (canceled)

41. (canceled)

42. (canceled)

43. (canceled)

44. (canceled)
